SEQUENCE LISTING (18 sequences)

SEQ ID NO: 1 - DNA, 22 bp, Artificial
  Primer 1
tcgagcggccgcccgggcaggt

SEQ ID NO: 2 - DNA, 20 bp, Artificial
  Primer 2R
agcgtggtcgcggccgaggt

SEQ ID NO: 3 - DNA, 21 bp, Artificial
  Forward primer
attgcacagtcaactaatttg

SEQ ID NO: 4 - DNA, 22 bp, Artificial
  Reverse primer
aaagttagccaattcaaaatta

SEQ ID NO: 5 - DNA, 17 bp, Artificial
  Forward primer
catggatgggaaggagc

SEQ ID NO: 6 - DNA, 17 bp, Artificial
  Reverse primer
gctgctggcacctaaag

SEQ ID NO: 7 - DNA, 17 bp, Artificial
  Forward primer
tagtcaccgaggtggtg

SEQ ID NO: 8 - DNA, 19 bp, Artificial
  Reverse primer
gctttcctccagaaatctc

SEQ ID NO: 9 - DNA, 17 bp, Artificial
  Forward primer
tcttcccaggctgtgag

SEQ ID NO: 10 - DNA, 19 bp, Artificial
  Reverse primer
ggtcaccagcttgttcttc

SEQ ID NO: 11 - DNA, 19 bp, Artificial
  Forward primer
cgatcatgtctggtgaggc

SEQ ID NO: 12 - DNA, 17 bp, Artificial
  Reverse primer
agcaccttcctcctttg

SEQ ID NO: 13 - DNA, 18 bp, Artificial
  Forward primer
cggcttcagagaagattg

SEQ ID NO: 14 - DNA, 19 bp, Artificial
  Reverse primer
gtaccgtttgtcaacctgc

SEQ ID NO: 15 - DNA, 20 bp, Artificial
  Forward primer
ttgcagaattccatgatctg

SEQ ID NO: 16 - DNA, 18 bp, Artificial
  Reverse primer
agaaggcttgtcctgatg

SEQ ID NO: 17 - DNA, 16 bp, Artificial
  Forward primer
cctgcactgacccttg

SEQ ID NO: 18 - DNA, 17 bp, Artificial
  Reverse primer
gtggctcagggtctttc

(19) United States
(12) Patent Application Publication
(10) Pub. No.: US 2008/0057498 A1
(43) Pub. Date: Mar. 6, 2008

(54) Differences in intestinal gene expression profiles

(76) Inventors: Theodoor Abram Niewold, Dronten (NL); Johanna Marina Jacoba Rebel, Lelystad (NL); Marinus Adrianus Smits, Harderwijk (NL)

Correspondence Address: TRASK BRITT, P.O. BOX 2550, SALT LAKE CITY, UT, 84110, US

(21) Appl. No.: 11/651,431

(22) Filed: Jan. 9, 2007

Related U.S. Application Data
(63) Continuation of application No. PCT/NL05/00494, filed on Jul. 8, 2005.

(30) Foreign Application Priority Data
Feb. 16, 2005 (EP) 05075373.0
Jul. 9, 2004 (EP) 04077001.8

(51) Int. Cl. C12Q 1/68; C07H 21/04

(52) U.S. Cl. 435/6; 536/23.2; 536/23.5; 536/24.33

(57) ABSTRACT

The invention provides a set of genes or gene sequences comprising at least two genes and the use of the set of genes or gene sequences for the determination of intestinal health and/or disease of an animal or a human. The invention further provides methods to detect the presence or absence of an intestinal disease in an animal or a human comprising measuring, in a sample of the animal or human, expression levels of a set of genes or gene sequences according to the invention, or a gene-specific fragment of the genes and comparing the expression levels with a reference value, such as the expression levels of the set of genes in a sample of intestinal tissue of a healthy animal or human.

### CROSS-REFERENCE TO RELATED APPLICATIONS

This application is a continuation of PCT International Patent Application No. PCT/NL2005/000494, filed on Jul. 8, 2005, designating the United States of America, and published, in English, as PCT International Publication No. WO 2006/006853 A2 on Jan. 19, 2006, which application claims priority to European Patent Application No. 05075373.0 filed Feb. 16, 2005, and European Patent Application No. 04077001.8 filed Jul. 9, 2004, the contents of the entirety of each of which are hereby incorporated herein by this reference.

### TECHNICAL FIELD

The invention relates to the field of biotechnology and diagnosis, more specifically to gene array diagnosis, even more specifically, to a set of differentially expressed genes, and measuring gene expression of the set of genes, in particular for assessing the health status of the intestinal mucosa and for assessment of alterations in the intestinal tract. The invention further relates to measuring gene expression of a set of genes for the evaluation of susceptibility to disease and the evaluation of the effect of food compounds and of oral pharmaceutical compounds or compositions on the intestinal tract.

### BACKGROUND

Examination of the host gene expression response to pathogens or noxious substances provides insight into the events that take place in the host. In addition, it sheds light on the basic mechanisms underlying differences in the susceptibility of the host to certain pathogens, noxious substances, or therapeutic substances. Many pathogens and many food and pharmaceutical compounds are tested in animals before admission for use in man. Better insight in the pathophysiology and pathology of the animals used in such experiments is important for the interpretation of the results and the translation of the results from the animal model to man. An important evaluation of animal experiments used to be the histopathological evaluation of animals sacrificed during or after an in vivo experiment.

Recently, genome sequencing projects and the development of DNA array techniques have provided new tools that provide a more comprehensive picture of the gene expression underlying disease states. For genome-wide gene expression analysis, serial analysis of gene expression (“SAGE”), differential display techniques, and both cDNA-based and oligonucleotide array-based technologies have been recently applied. Oligonucleotide- or cDNA-based arrays have proven to be useful for the analysis of multiple samples (Dieck1).

Genome-wide gene expression analysis of tissue samples from affected and normal individuals of one species illuminate important events involved in disease pathogenesis. For example, in inflammatory bowel diseases like, for example, Crohn's disease or Ulcerative Colitis, individual mRNAs serve as sensitive markers for recruitment and involvement of specific cell types, cellular activation, and mucosal expression of key immunoregulatory proteins. Disease heterogeneity, reflecting differences in underlying environmental and genetic factors leading to the inflammatory mucosal phenotype, is reflected in different gene expression profiles. Most reported GeneChip or microarray studies have centered on cultured cell lines or purified single cell populations.

The measurement and analysis of gene expression in diseases involving more complex tissues, such as the intestine, pose several unique challenges and is very difficult to interpret. The inflammatory mucosa is composed of heterogeneous and changing cell populations. Furthermore, the interactions of immune cell populations with non-immune cellular components of the intestinal mucosa, including epithelial, mesenchymal, and microvascular endothelial cells, are thought to be pivotal in the pathogenesis of inflammatory bowel disease.

Gene expression measurements of a sample of the gastrointestinal tract were considered to be inaccurate because such a sample often represents an average of these many different cell types. As a result of mucosal trafficking of inflammatory cell populations, for instance, in inflammatory bowel disease, gene expression by a certain cell population (e.g., epithelial cells) is decreased relative to the total mRNA pool. Meaningful gene expression differences are also often hidden in genetic noise or complex patterns of mucosal gene expression unrelated to disease pathogenesis.

### DISCLOSURE OF THE INVENTION

Provided is a method for determining the presence or absence of an intestinal disease that is independent of the specific kind of disease and independent of the species of the animal. Also provided is a set of genes or gene sequences. At least five of these genes or gene sequences are used in order to obtain an expression pattern that is indicative for the intestinal health status of an animal or human.

Compared are the results of studies on intestinal alterations in different animals and with different pathogens or noxious substances, to select a set of genes that is highly predictive for intestinal health. Therefore, studies were undertaken to examine the utility of gene expression profiling combined with sophisticated gene clustering analyses to detect distinctive gene expression patterns that associate with histological score and clinical features of damaged integrity of the intestinal mucosa of chickens and of pigs. Studies in different chicken lines with a varying susceptibility to Malabsorption Syndrome (“MAS”) and in chicken lines with a different susceptibility to Salmonella bacteria were compared with studies in an ex vivo experimental set-up testing different pathogens like, for example, E. coli, rotavirus and salmonella bacteria in intestinal mucosa of live pigs.

Surprisingly, it was found that a common expression profile of a subset of genes is indicative of intestinal health, both in chickens and pigs. The same subset of genes that were up- or down-regulated in the chicken model with MAS infection, were also found to be up- or down-regulated in porcine intestines after damaging the integrity of the mucosa of the intestinal tract. This means that the set of genes disclosed in this specification in Table 1 is indicative of intestinal health in animal species as different as mammals and birds. Therefore, the invention provides a set of genes indicative of intestinal health, which is not restricted to an animal species.

### BRIEF DESCRIPTION OF THE DRAWINGS

FIG. 1: Differential gene expression between normal and enteropathogenic E. coli infected intestinal loops (animal 6). Scatter plot displaying the mean expression profile of all genes represented on the microarray, based on two slides. Points above the +2 or below the −2 line represent significant differences.

FIG. 2: Expression of I-FABP and PAP as established by microarray (m) and Northern blot (nb).

FIG. 3: Amount of CFU of Salmonella Enteritidis in the liver of chickens from the susceptible and resistant chicken lines (n=5).

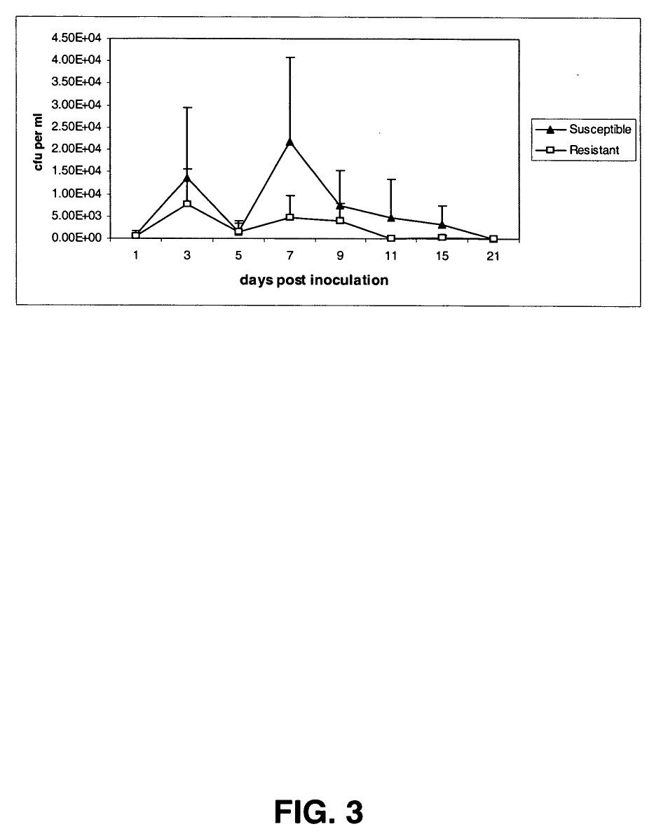

FIG. 4: Percentage growth of broilers infected with 105 Salmonella Enteritidis compared to healthy counterparts (n=5). S=susceptible chicken line; R=resistant chicken line.

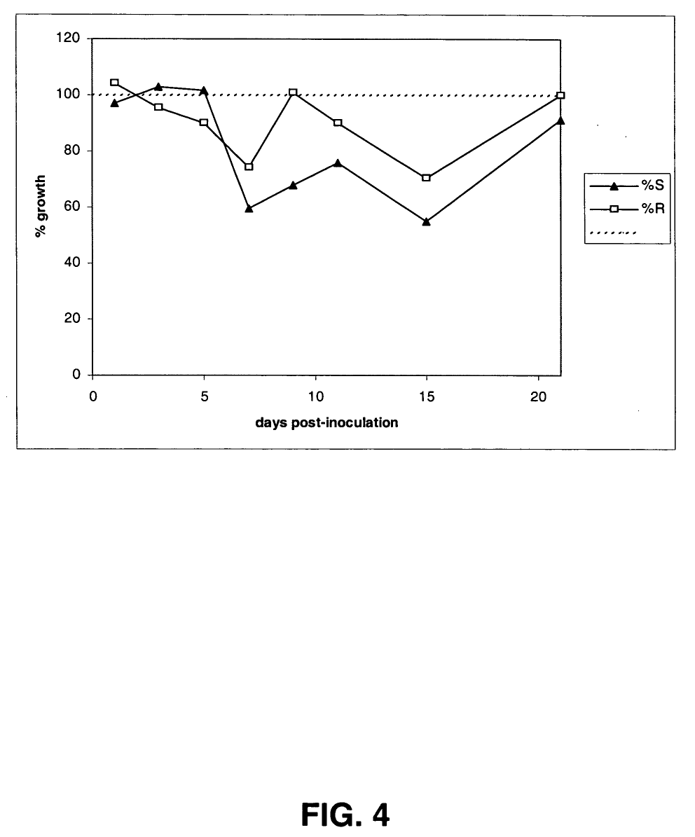

### DETAILED DESCRIPTION OF THE INVENTION

TABLE 1
Genes differentially expressed during alteration of the intestinal
mucosa

| | Homology with | | | Chicken |
|---|---|---|---|---|
| Gene name | Accession No. | Chicken | Pig | and pig |
| Na/glucose transporter | gi: 12025666 | yes* | yes | yes |
| K/Cl channel | gi: 5174550 | yes | yes | yes |
| I-FABP | gi: 10938019 | yes | yes | yes |
| L-FABP | | yes | yes | yes |
| Cytochrome P450 | gi: 1903316 | yes | yes | yes |
| Caspase | | yes | yes | yes |
| Beta-2-microglobin | | yes | yes | yes |
| Guanylyn | XM_424439.1 | yes | yes | yes |
| Calbindin | NM_205513 | yes | yes | yes |
| Phosphatase | | yes | yes | yes |
| Aldolase | | yes | yes | yes |
| Actin | gi: 57977284 | yes | yes | yes |
| metalloproteinase | gi: 54112079 | yes | yes | yes |
| Aminopeptidase | | yes | yes | yes |
| glycosaminotransferase | | yes | yes | yes |
| glutathion S transferase | | yes | yes | yes |
| maltase/glucoamylidase | | yes | yes | yes |
| sucrase/isomaltase | | yes | yes | yes |
| Butyrophilin | XM_4164021 | yes | yes | yes |
| ApoB | gi: 178817 | yes | yes | yes |
| Cytochrome C oxidase | | yes | yes | yes |
| Pancreatitis associated protein | . | | yes | |
| beta-1,6-N-glucosaminyltransferase | gi: 32396225 | yes | yes | yes |
| THO transcriptie enhancer | | | yes | |
| STAT | gi: 47080105 | yes | yes | yes |
| Phosphodiesterase | | | yes | |
| SRC-like tyrosine kinase | XM_418206.1 | | yes | |
| Hensin | | yes | | |
| SGLT-1 | | yes | yes | yes |
| zinc-binding protein | | yes | | |
| aldo-ketoreductase | | yes | | |
| retinol-binding protein | | yes | | |
| Pyrin | | yes | | |
| Meprin | | yes | | |
| Apo A | | yes | | |
| Gastropin | | yes | | |
| CD3 epsilon (CD3E) | NM_206904.1 | yes | | |
| PREDICTED: similar to novel | XM_414886.1 | yes | | |
| interleukin receptor | | | | |
| PREDICTED: similar to signal | XM_421900.1 | yes | yes | yes |
| transducer and activator of transcription | | | | |
| 4 (STAT4) | | | | |
| T-cell receptor beta chain constant | AF110982.1 | yes | | |
| region | | | | |
| PREDICTED: similar to T-cell | XM_416744.1 | yes | similarity | |
| ubiquitin ligand protein TULA short | | | | |
| form | | | | |
| CDH1-D | AF421549 | yes | | |
| PREDICTED: similar to eukaryotic | XM_423296.1 | yes | similarity | |
| translation initiation factor 4 gamma, 3 | | | | |
| (eIF4g) | | | | |
| PREDICTED: similar to normal | XM_413822.1 | yes | | |
| mucosa of esophagus specific 1 | | | | |
| gene 37LRP/p40 | X94368 | yes | | |
| initiation factor 5A (eIF5A) | NM_205532.1 | yes | similarity | |
| PREDICTED: similar to | XM_422123.1 | yes | similarity | |
| insulin-induced protein 2; INSIG2 | | | | |
| membrane protein | | | | |
| PREDICTED: similar to MGC52743 | XM_420146.1 | yes | | |
| protein | | | | |
| G. gallus mRNA for iodothyronine | Y11273.1 | yes | | |
| deiodinase type III | | | | |
| finished cDNA, clone ChEST518c13 | CR405893.1 | yes | | |
| PREDICTED: similar to Kelch-like | XM_422912.1 | yes | | |
| protein 5 | | | | |
| PREDICTED: similar to G-protein | XM_425740.1 | yes | | |
| coupled receptor | | | | |
| ribosomal protein L13 (RPL13) | NM_204999.1 | yes | | |
| spermidine/spermine | NM_204186.1 | yes | similarity | |
| N1-acetyltransferase (SSAT) | | | | |
| PREDICTED: similar to | XM_416148.1 | yes | | |
| NADH: ubiquinone oxidoreductase | | | | |
| b17.2 subunit | | | | |
| cytochrome P450 A 37 (CYP3A37) | NM_001001751.1 | yes | similarity | |
| apoB mRNA encoding apolipoprotein | M18421 | yes | similarity | |
| finished cDNA, clone ChEST46a1 | CR353265.1 | yes | | |
| PREDICTED: Gallus gallus similar to | XM_422715 | yes | | |
| Fc fragment of IgG binding protein; IgG | | | | |
| Fc binding protein | | | | |
| Gallus gallus RhoA GTPase (RHOA), | NM_204704.1 | yes | | |
| mRNA | | | | |
| PREDICTED: similar to | XM_421662.1 | yes | | |
| Interferon-induced protein with | | | | |
| tetratricopeptide repeats 5 (IFIT-5) | | | | |
| (Retinoic acid- and interferon-inducible | | | | |
| 58 kDa protein) | | | | |
| Gallus gallus finished cDNA, clone | CR352925.1 | yes | | |
| ChEST402p8 | | | | |
| PREDICTED: similar to proprotein | XM_424712.1 | yes | | |
| convertase subtilisin/kexin type 1 | | | | |
| preproprotein; prohormone convertase | | | | |
| 3; prohormone convertase 1; | | | | |
| neuroendocrine convertase 1; proprotein | | | | |
| convertase 1 | | | | |
| Gallus gallus protein tyrosine | NM_204417.1 | yes | | |
| phosphatase, receptor type, C (PTPRC) | | | | |
| PREDICTED: similar to archease | XM_417810 | yes | | |
| Gallus gallus similar to ARHGAP15 | NM_001008476.1 | yes | | |
| casein kinase II alpha subunit | NM_001002242 | yes | | |
| PREDICTED: similar to tumor necrosis | XM_417585 | yes | | |
| factor receptor superfamily, member 18 | | | | |
| isoform 3 precursor | | | | |
| similar to Psmc6 protein | NM_001006494 | yes | | |
| lactate dehydrogenase H subunit | AF069771 | yes | | |
| (LDH-B) | | | | |
| PREDICTED: similar to T-cell | XM_419701 | yes | | |
| activation Rho GTPase-activating | | | | |
| protein isoform b | | | | |
| eukaryotic translation elongation factor | NM_204157 | yes | | |
| 1 alpha 1 | | | | |
| similar to Gps1 | NM_001006206 | yes | | |
| mRNA for hypothetical protein, clone | AJ719784 | yes | | |
| 6h13 | | | | |
| PREDICTED: similar to RasGEF | XM_421515 | yes | | |
| domain family | | | | |
| alpha-3 collagen type VI | NM_205534 | yes | | |
| TRAF-5 mRNA for tumor necrosis | AB100868 | yes | | |
| factor receptor associated factor-5 | | | | |
| PREDICTED: similar to Rac2 protein | XM_416280 | yes | | |
| Rel-associated pp40 | NM_001001472 | yes | | |
| PREDICTED: similar to | XM_422360 | yes | similarity | |
| calcium-activated chloride channel | | | | |
| PREDICTED: Gallus gallus similar to | XM_425603.1 | yes | | |
| ORF2 | | | | |
| PREDICTED: similar to inducible | XM_421959.1 | yes | | |
| T-cell co-stimulator | | | | |
| PREDICTED: similar to | XM_420925 | yes | | |
| interferon-induced membrane protein | | | | |
| Leu-13/9-27 | | | | |
| PREDICTED: similar to Rho | XM_423002.1 | yes | | |
| GTPase-activating protein; | | | | |
| brain-specific RhoGTP-ase-activating | | | | |
| protein; racGTPase-activating protein; | | | | |
| GAB-associated CDC42; RhoGAP | | | | |
| involved in the catenein-N-cadherin and | | | | |
| NMDA receptor signaling | | | | |
| Gallus gallus mRNA for | AJ006405 | yes | | |
| glutathione-dependent prostaglandin-D | | | | |
| synthase | | | | |
| GGIKTRF G. gallus mRNA for Ikaros | Y11833.1 | yes | | |
| transcription factor | | | | |
| PREDICTED: similar to protein | XM 417797.1 | yes | | |
| tyrosine phosphatase 4a2 | | | | |
| PREDICTED: Gallus gallus similar to | XM 417652.1 | yes | | |
| guanylin precursor (LOC419498) | | | | |
| PREDICTED: Gallus gallus similar to | XM_416896.1 | yes | | |
| lysozyme (EC 3.2.1.17) g [validated] - | | | | |
| goose (LOC418700) | | | | |
| Homo sapiens signal transducer and | gi: 47080105 | similarity | yes | |
| activator of transcription 3 (acute-phase | | | | |
| response factor) (STAT3) | | | | |
| Sus scrofa triadin gene | gi: 15027104 | | yes | |
| Canis familiaris multidrug resistance | gi: 2852440 | | yes | |
| p-glycoprotein mRNA | | | | |
| Bos Taurus calpastatin mRNA | gi: 5442419 | | yes | |
| Sus scrofa myostatin gene, complete cds | gi: 34484364 | | yes | |
| Sus scrofa calbindin D-9k mRNA | gi: 294215 | similarity | yes | |
| Homo sapiens cDNA FLJ11576 fis, | gi: 10432858 | | yes | |
| clone HEMBA1003548 | | | | |
| Homo sapiens fatty acid binding protein | gi: 10938019 | similarity | yes | |
| 2, intestinal (FABP2), mRNA | | | | |
| S. scrofa mRNA for glutathione | gi: 1185279 | similarity | yes | |
| S-transferase | | | | |
| Homo sapiens chloride channel, calcium | gi: 12025666 | similarity | yes | |
| activated, family member 4 | | | | |
| Sus scrofa Pancreatic secretory trypsin | gi: 124857 | | yes | |
| inhibitor | | | | |
| Homo sapiens transmembrane 4 L six | gi: 13376165 | | yes | |
| family member 20(TM4SF20) | | | | |
| Sus scrofa thioredoxin mRNA | gi: 14326452 | | yes | |
| Homo sapiens ribosomal protein L23 | gi: 14591907 | | yes | |
| (RPL23), mRNA | | | | |
| Porcine D-amino acid oxidase mRNA | gi: 164305 | | yes | |
| Pig Na+/glucose cotransporter protein | gi: 164674 | | yes | |
| (SGLT1) mRNA | | | | |
| Rabbit mRNA for neutral endopeptidase | gi: 1651 | | yes | |
| (NEP) | | | | |
| Oryctolagus cuniculus | gi: 165800 | | yes | |
| UDP-glucuronosyltransferase | | | | |
| (UGT2C1) mRNA | | | | |
| Vitamin D-dependent calcium-binding | gi: 1710817 | | yes | |
| protein, intestinal (CABP) | | | | |
| Homo sapiens cell division cycle 42 | gi: 17391364 | | yes | |
| (GTP binding protein, 25 kDa) | | | | |
| Homo sapiens I factor (complement), | gi: 18089116 | | yes | |
| mRNA | | | | |
| Homo sapiens guanylate binding protein | gi: 18490137 | | yes | |
| 2, interferon-inducible | | | | |
| Human pancreatitis associated protein | gi: 189600 | | yes | |
| mRNA (PAP), complete cds (= Bovine | | | | |
| PTP; gi \|18767559\|) | | | | |
| S. scrofa CYP3A29 mRNA for | gi: 1903316 | similarity | yes | |
| cytochrome P450 | | | | |
| Pig mRNA for haptocorrin | gi: 1963 | | yes | |
| Homo sapiens transmembrane | gi: 20381190 | | yes | |
| channel-like 5, mRNA | | | | |
| Human L1 element L1.25 p40 and | gi: 2072970 | | yes | |
| putative p150 genes, complete cds | | | | |
| Homo sapiens tyrosine | gi: 21464103 | | yes | |
| 3-monooxygenase/tryptophan | | | | |
| 5-monooxygenaseactivation protein, | | | | |
| theta polypeptide (YWHAQ), mRNA | | | | |
| Similar to Homo sapiens OCIA domain | gi: 21619772 | | yes | |
| containing 2, mRNA | | | | |
| Homo sapiens cDNA FLJ40597 fis, | gi: 21757819 | | yes | |
| clone THYMU2011118 | | | | |
| centromere/kinetochore protein (Zw10), | gi: 22165348 | | yes | |
| mRNA | | | | |
| Homo sapiens proteasome (prosome, | gi: 23110943 | | yes | |
| macropain) subunit, alpha type, 6 | | | | |
| Homo sapiens glucosamine | gi: 25059057 | | yes | |
| (N-acetyl)-6-sulfatase | | | | |
| Homo sapiens keratin 20, mRNA | gi: 27894336 | | yes | |
| Homo sapiens muscleblind-like | gi: 28175587 | | yes | |
| (Drosophila), mRNA | | | | |
| Human mRNA for aldolase B | gi: 28616 | similarity | yes | |
| Homo sapiens ribonuclease L, mRNA | gi: 30795246 | | yes | |
| aldehyde dehydrogenase 1 family, | gi: 31342530 | | yes | |
| member A1 | | | | |
| Homo sapiens olfactomedin 4 | gi: 32313592 | | yes | |
| (OLFM4), mRNA (GW112 mRNA) | | | | |
| lactase-phlorizin hydrolase gene | gi: 32481205 | | yes | |
| Bos taurus carcinoembryonic | gi: 33638079 | | yes | |
| antigen-related cell adhesion molecule 1 | | | | |
| isoform 3Ss (CEACAM1) mRNA | | | | |
| Homo sapiens eukaryotic translation | gi: 33877073 | similarity | yes | |
| initiation factor 3, subunit 1 | | | | |
| Homo sapiens clone DNA58855 | gi: 37182463 | | yes | |
| TCCE518 (UNQ518) mRNA | | | | |
| Macaca mulatta actin beta subunit | gi: 38112260 | similarity | yes | |
| (ACTB) mRNA | | | | |
| Homo sapiens DKFZp564J157 protein, | gi: 39644474 | | yes | |
| mRNA | | | | |
| Homo sapiens hypothetical protein | gi: 40254892 | | yes | |
| FLJ11273 (FLJ11273) | | | | |
| Homo sapiens hypothetical LOC148280 | gi: 41058029 | | yes | |
| mRNA | | | | |
| Sus scrofa mRNA for hypothetical | gi: 41058029 | | yes | |
| protein | | | | |
| Sus scrofa mRNA for hypothetical | gi: 4186144 | | yes | |
| protein | | | | |
| Homo sapiens disabled homolog 2, | gi: 4503250 | | yes | |
| mitogen-responsive phosphoprotein | | | | |
| (Drosophila) (DAB2) | | | | |
| Homo sapiens hydroxysteroid (17-beta) | gi: 4504502 | | yes | |
| dehydrogenase 2 | | | | |
| Homo sapiens insulin-like growth factor | gi: 4504610 | similarity | yes | |
| 2 receptor (IGF2R), mRNA | | | | |
| S. scrofa mRNA for liver fatty acid | gi: 455524 | similarity | yes | |
| binding protein | | | | |
| Homo sapiens hypothetical protein | gi: 46195796 | | yes | |
| LOC51321 (LOC51321), mRNA | | | | |
| Sus scrofa ASIP gene for agouti | gi: 46240693 | | yes | |
| signaling protein and AHCY gene for | | | | |
| S-adenosylhomocysteine hydrolase | | | | |
| Sus scrofa interferon gamma (IFNG), | gi: 47522725 | | yes | |
| mRNA | | | | |
| Sus scrofa mRNA for caspase-3 | gi: 47523065 | similarity | yes | |
| Sus scrofa alveolar macrophage-derived | gi: 47523123 | | yes | |
| chemotactic factor-I mRNA/IL8 | | | | |
| Sus scrofa microsomal triglyceride | gi: 47523449 | | yes | |
| transfer protein large subunit (MTP), | | | | |
| mRNA | | | | |
| Sus scrofa spermidine/spermine | gi: 47523773 | similarity | yes | |
| N-acetyltransferase (SAT) | | | | |
| Sus scrofa methylmalonyl-CoA mutase | gi: 47523863 | | yes | |
| (MUT), mRNA | | | | |
| Homo sapiens Nipped-B homolog | gi: 47578106 | | yes | |
| (Drosophila) (NIPBL), transcript variant | | | | |
| B, mRNA | | | | |
| Homo sapiens maltase-glucoamylase | gi: 4758711 | | yes | |
| (alpha-glucosidase) (MGAM), mRNA | | | | |
| Homo sapiens RNA-binding protein, | gi: 48735253 | | yes | |
| mRNA | | | | |
| Homo sapiens ubiquitin D (UBD), | gi: 50355987 | similarity | yes | |
| mRNA | | | | |
| Homo sapiens glutaryl-Coenzyme A | gi: 50959149 | | yes | |
| dehydrogenase (GCDH) | | | | |
| S. scrofa mRNA for aminopeptidase N | gi: 525286 | | yes | |
| Interstitial collagenase precursor | gi: 54112079 | similarity | yes | |
| (Matrix metalloproteinase-1) (MMP-1) | | | | |
| Homo sapiens topoisomerase-related | gi: 5565688 | | yes | |
| function protein (TRF4-2) mRNA | | | | |
| Canis familiaris similar to seven | gi: 57085092 | | yes | |
| transmembrane helix receptor | | | | |
| (LOC479238) | | | | |
| Canis familiaris similar to | gi: 57097500 | | yes | |
| phospholipases inhibitor (LOC482701), | | | | |
| mRNA | | | | |
| weakly similar to rattus norvegicus | gi: 7407646 | | yes | |
| hyperpolarization-activated, cyclic | | | | |
| nucleotide-gated potassium channel 2 | | | | |
| (HCN2) mRNA | | | | |
| Homo sapiens uncharacterized bone | gi: 7688976 | | yes | |
| marrow protein BM041 mRNA | | | | |
| Homo sapiens THO complex 4 | gi: 55770863 | | yes | |
| (THOC4) | | | | |
| Human apolipoprotein B-100 mRNA, | gi\|178817 | similarity | yes | |
| complete cds | | | | |
| Homo sapiens clone DNA59613 | gi\|37182060 | | yes | |
| phospholipase inhibitor (UNQ511) | | | | |
| mRNA | | | | |
| Danio rerio glutamate-cysteine ligase, | gi\|41054138 | | yes | |
| modifier subunit (gclm) | | | | |
| Sus scrofa ribophorin I | gi\|9857226 | | yes | |
| Homo sapiens beta | gi\|9910143 | | yes | |
| 1,3-galactosyltransferase (C1GALT1), | | | | |
| mRNA | | | | |

*= the expression level of genes is at least two-fold increased or decreased compared to control values

Table 1 demonstrates that there are a number of common genes differentially expressed in chickens and in pigs after damaged integrity of the intestinal mucosa. Because the same subset of responsive genes is found in two such different animal species as the pig and the chicken after alteration of the gut mucosa by viral or bacterial cause, this set of the last column of Table 1 has a strong predictive value for damage to the intestinal mucosa.

Hence, in one aspect, the invention provides a set of genes or gene sequences comprising at least five genes selected from the following genes: Na/glucose transporter (SGLT1), K/Cl channel, I-FABP, L-FABP, Cytochrome P450, caspase, Beta-2-microglobin, guanylyn, calbindin, phosphatase, aldolase, (beta-)actin, metalloproteinase, aminopeptidase, (acetyl)glycosaminotransferase, glutathion S transferase, maltase/glucoamylidase, sucrase/isomaltase, butyrophilin, apoB, and cytochrome C oxidase.

In another aspect, the invention provides a set of genes or gene sequences comprising at least five genes selected from the following genes: Na/glucose transporter (SGLT1), K/Cl channel, I-FABP, L-FABP, Cytochrome P450, caspase, Beta-2-microglobin, guanylyn, calbindin, phosphatase, aldolase, (beta-)actin, metalloproteinase, aminopeptidase, (acetyl) glycosaminotransferase, glutathion S transferase, maltase/glucoamylidase, sucrase/isomaltase, butyrophilin, apoB, cytochrome C oxidase, and STAT3 and STAT4.

Taking into account that a large evolutionary distance exists between chickens and pigs, and that there are differences between the challenge methods (MAS virus like, E. coli, salmonella, rotavirus), it is unexpected that the same subset of genes is reactive as a result of intestinal mucosal disease or degeneration. A method of diagnosing intestinal disease or monitoring intestinal health has been provided, comprising measuring, in a sample of an animal or human, expression levels of a set of genes or gene sequences according to the invention, or a gene-specific fragment of the genes and comparing the expression levels with a reference value.

A method of the invention is suitable for such a vast array of animals as birds and mammals, including man.

A method of the invention is also suitable for evaluating the beneficial or the negative effect of certain food or pharmaceutical components on the intestines.

In another embodiment, a method of the invention is used to determine the susceptibility of a human, or an animal, or a breed of animals for a certain pathogen or a food or pharmaceutical component. Of course, it is not necessary to determine the differential expression level of all genes mentioned in the last column of Table 1. Therefore, the invention discloses a set of genes or gene sequences comprising at least five genes selected from the last column of Table 1.

In a more preferred embodiment, at least two of the genes are comprised in the set of genes.

Further experimentation has shown that the gene set preferably comprises at least five genes of the following nine genes: Na/glucose transporter (SGLT1), Ca/Cl channel, FABP, Cytochrome P450, (beta-)actin, acetylglycosaminyltransferases, Meprin A, apoB, and STAT.

Even more preferably, the set of genes comprises six genes of the following nine genes: Na/glucose transporter (SGLT1), Ca/Cl channel, FABP, Cytochrome P450, (beta-)actin, acetylglycosaminyltransferases, Meprin A, apoB, and STAT.

In an even more preferred embodiment, the set of genes comprises seven, or eight or nine genes of the following nine genes: Na/glucose transporter (SGLT1), Ca/Cl channel, FABP, Cytochrome P450, (beta-)actin, acetylglycosaminyltransferases, Meprin A, apoB, and STAT.

“Differential gene expression” in this application means that the level of mRNA and/or protein is significantly increased or decreased as compared to a reference value. Preferably, the level of mRNA and/or protein is at least two-fold increased or decreased compared to a reference value. The reference value in one embodiment comprises the level of the same or a comparable mRNA and/or protein of a tissue sample of a control animal. In one embodiment, the differential expression affects a protein product and/or the (enzymatic) activity (or parts thereof) of the genes.

The term “control animal” preferably comprises an animal of the same species and about the same age, which has not been subjected to the alterations in the intestinal tract, or an animal of the same species and about the same age, but from a resistant breed.

The term “control” preferably comprises the same kind of sample of an animal of the same species and age or to the same kind of sample of the same animal, the sample not being affected with the alterations in the intestinal tract. The control sample is, for example, taken prior to the alteration of the mucosa.

Now that a set of genes is disclosed that enables the diagnosis of intestinal health and/or disease, this information is used in one embodiment for the determination of intestinal health and/or disease of an animal or human, preferably under normal living conditions and preferably also under experimental conditions. Therefore, in one embodiment of the invention, a use of a set of genes or gene sequences according to the invention for the determination of intestinal health and/or disease of an animal or a human is provided, as well as a method of detecting the presence or absence of an intestinal disease in an animal comprising measuring, in a sample of intestinal tissue of the animal or human, expression levels of a set of genes or gene sequences according to the invention, or a gene-specific fragment of the genes and comparing the expression levels with the expression levels of the set of genes in a sample of intestinal tissue of a healthy animal or human.

The testing preferably occurs on a sample of intestinal tissue, but in another embodiment, the image of the same expression profile occurs in another sample, such as, for example, blood or intestinal contents, or other body effluent. Therefore, in one aspect, the invention provides a method of the invention, wherein the sample comprises a body sample of the animal or human. A body sample in this specification comprises, but is not restricted to, stool or intestinal contents, urine, blood, and sputum.

In another embodiment, repeated measurement of intestinal health gives information about the effect of certain measures or conditions with respect to dietary, housing or sanitary conditions. Therefore, the invention also discloses a method of measuring a change, preferably an increase, of the intestinal health status of an animal or human, comprising measuring in a series of samples of the animal or human taken at different time points, expression levels of a set of genes of the invention, or a functional equivalent or fragment of the genes, and comparing the expression levels of a reference value, such as an expression level of the genes in a sample of intestinal tissue of a healthy animal or human.

As mentioned before, it is not necessary to determine the differential expression level of all genes of the invention. Of course, now that genes that become differentially expressed after damage of the intestinal wall are disclosed in the invention, a skilled person can easily select some of these genes and adjust the set to his own liking. It is clear that the most reliable results will often be obtained by determining a larger number of differentially expressed genes, rather than determining a smaller number of genes, but the invention discloses that even the determination of five or two genes of the invention is enough to diagnose damage of the intestinal mucosa. Therefore, the invention discloses a method of measuring a change, preferably an increase, of the intestinal health status or the presence or absence of intestinal disease of an animal or human, comprising measuring expression levels of at least two genes of a set of genes of the invention, or a gene-specific fragment of the genes. More preferably, the differential expression of three, four, five, six, seven, eight, or nine genes is measured.

By a “gene-specific fragment of a gene of the invention” is meant a part of the nucleic acid of the gene at least 20 base pairs long, preferably at least 50 base pairs long, more preferably at least 100 base pairs long, even more preferably at least 150 base pairs long, and most preferably at least 200 base pairs long, comprising at least one binding site for a gene-specific complementary nucleic acid such as, for example, a gene-specific PCR primer.

In another embodiment, the invention also discloses a method of the invention comprising measuring expression levels of at least ten genes, or a combination of any of the genes according to the invention, or a gene-specific fragment of the genes. The invention also discloses a method as described before, comprising measuring expression levels of at least 20 genes, or a combination of any of the genes of the invention, or a gene-specific fragment of the genes.

Methods as described herein are especially suited for investigating the health or disease status of the intestine after administration of certain substances to an animal. Administration, preferably enteral administration, of a food compound, a pharmaceutical composition, a microorganism, or a pathogen, or part thereof, to an animal, and measuring, before and after administration, what changes occur in gene expression of at least two of the genes of the invention in response to the administration, will assess the health status of the intestines of the animal. Some aspects of the invention are also conducted in humans. Enteral administration in this application comprises the oral or intra-intestinal administration of a composition. Therefore, in another embodiment, the invention discloses a method of the invention wherein a compound is administered enterally to an animal or human.

In certain embodiments, the invention includes a method of the invention wherein the compound is a part of the food of the animal or human. In this way, the effects on the intestinal mucosa of a certain kind of food supplement, food additive, artificial and natural flavor and/or color, and/or any other molecule, is tested for its use in food of animals and/or humans. Of course, animal experiments are very useful to test the effects of the above-mentioned compounds on the intestine, but the ultimate proof of any substance that is added to human food is in the administration of the compounds to human volunteers. Therefore, the invention also discloses a method of the invention wherein the compound is a food compound or a part thereof. Determination of an effect on the intestine of a pathogenic compound and/or a virus and/or microorganism such as, for instance, parasites and bacteria, is also enabled by a method of the invention.

Also disclosed is a method of the invention, wherein a pathogenic compound or a part thereof, and/or a virus or a microorganism or a part thereof is administered, preferably enterally, to an animal or human. Also disclosed is a method, wherein a pharmaceutical composition or a part thereof is administered, preferably enterally, to an animal or human.

Also provided is a method of selecting an animal breed on the basis of their reaction pattern in the microarray after challenging the intestinal health status of an animal. By testing the intestinal health with a method of the invention under various conditions or after specific challenges with a virus or bacteria or other compound, a breeder is able to select a breed of animal that is better suited for production of animal products like, for example, milk, meat, or eggs. The animal breed is, therefore, better adapted to, for example, a high incidence of a certain pathogen or a specific component in the food that affects the intestinal health status of the animal breed. This knowledge also discloses to a breeder which genes and/or gene combinations are more suitable for a certain breeding line of an animal and, therefore, the invention discloses a tool for selecting a breeding line of an animal.

In certain embodiments, a certain breed of animals is subjected to a challenge infection with an intestinal pathogen, like is presented in the Examples. Comparing the microarray results of the challenged animals with those of control animals, or of challenged animals of a different breed, discloses which animal breed is susceptible and which breed is resistant to the pathogen.

The invention enables assessment of the health status of an animal or a human. Once the health status is defined, the health status is in one embodiment ameliorated, for instance, by administration of a food component, additive, microbial organism or component, and/or by a pharmaceutical composition. Therefore, the invention also provides a food component, food additive, microbial organism or component, and/or pharmaceutical composition selectable by a method of the invention and characterized in that they increase the intestinal health status.

In certain embodiments, the invention discloses a kit containing at least one ingredient to measure protein levels of at least two genes of the invention. The protein levels are preferably measured in a bodily sample as defined in this application.

In another embodiment, the invention discloses a kit comprising a set of at least two primers capable of specifically hybridizing to at least two nucleic acid sequences encoding any one of the genes of Table 1, or a gene-specific fragment of the genes. In certain embodiments, the genes are of porcine origin, more preferably, the genes are of avian origin, even more preferably, the genes are of bovine origin, and most preferably, the genes are of human origin.

In certain embodiments, a method according to the invention is used to estimate the intestinal health status of a pig or a chicken. More preferably, the intestinal health status of a pig infected with E. coli, salmonella, rotavirus, or a combination thereof, is determined, or the intestinal health status of a chicken infected with MAS, salmonella, or a combination thereof is determined. Preferably, use is made of at least five genes of the following nine genes: Na/glucose transporter (SGLT1), Ca/Cl channel, FABP, Cytochrome P450, (beta-)actin, acetylglycosaminyltransferase Meprin A, apoB, and STAT.

The invention is further described with the aid of the following illustrative Examples.

### EXAMPLE 1

### Differences in Intestinal Gene Expression Profiles in Broiler Lines Varying in Susceptibility to Malabsorption Syndrome

Here, the research results are described on the transcriptional response in the intestine of broilers after a MAS induction and on the difference in gene expression and MAS susceptibility. Gene expression differences in the intestine were investigated using a cDNA microarray containing more than 3000 EST derived from a normalized and subtracted intestinal cDNA library (van Hemert, Ebbelaar et al., 2003). The findings were confirmed using a quantitative RT-PCR.

Materials and Methods

Chickens

Two broiler lines, S (“susceptible”) and R (“resistant”), were used in the present study (Nutreco®, Boxmeer, NL). They were described earlier as B and D, respectively (Zekarias, Songserm et al., 2002). Sixty one-day-old chicks of each line (S and R) were randomly divided into two groups, 30 chicks each. One group was orally inoculated with 0.5 ml of the MAS-homogenate (homogenate C in (Songserm et al., 2000)) and the other was the control group, orally inoculated with 0.5 ml Dulbecco's phosphate buffered saline (PBS). Five chicks of each group were randomly chosen and sacrificed at eight hours, day 1, 3, 5, 7 and 11 post-inoculation (pi) and tissue samples were collected. Pieces of the jejunum were snap frozen in liquid nitrogen and kept at −70° C. until further use. Adjacent parts of the jejunum were fixed in 4% formaldehyde and used for histopathology and immunohistochemistry. The study was approved by the institutional Animal Experiment Commission in accordance with the Dutch regulations on animal experimentation.

The same set-up, lines and groups were used for a second animal experiment, although in that experiment, three chicks of each group were sacrificed at day 1, 3 and 13 post-inoculation. The same tissues were sampled.

RNA Isolation

Pieces of the jejunum were crushed under liquid nitrogen. Fifty to 100 mg tissues of the different chicks were used to isolate total RNA using TRIzol reagent (GibcoBRL), according to instructions of the manufacturer with an additional step. The homogenized tissue samples were solved in 1 ml of TRIzol Reagent using a syringe and needle 21G passing the lysate ten times. After homogenization, insoluble material was removed from the homogenate by centrifugation at 12,000×g for ten minutes at 4° C.

For the array, hybridization pools of RNA were made in which equal amounts of RNA from the different chickens of the same line, condition and time point were present.

Hybridizing of the Microarray

The microarrays were constructed as described earlier and contained 3072 cDNAs spotted in duplicate (van Hemert, Ebbelaar et al., 2003). Before hybridization, the microarray was pre-hybridized in 5% SSC, 0.1% SDS and 1% BSA at 42° C. for 30 minutes. To label the RNA, a MICROMAX TSA labeling and detection kit (PerkinElmer) was used. The TSA probe labeling and array hybridization were performed as described in the instruction manual with minor modifications. Biotin- and fluorescein-labeled cDNAs were generated from 5 μg of total RNA from the chicken jejunum pools per reaction. The cDNA synthesis time was increased to three hours at 42° C., as suggested (Karsten et al., 2002). Post-hybridization washes were performed according to the manufacturer's recommendations. Hybridizations were repeated with the fluorophores reversed. After signal amplification, the microarrays were dried and scanned in a GeneTAC2000 (Genomic Solutions). The image was processed (geneTAC software, Genomic Solutions) and spots were located and integrated with the spotting file of the robot. Reports were created of total spot information and spot intensity ratio for subsequent data analyses.

Analysis of the Microarray Data

After background correction, the data presented in an M/A plot were M=log₂R/G and A=log₂√(R×G) (Dudoit et al., 2002). An intensity-dependent normalization was performed using the lowest function in the statistical software package R (Yang et al., 2002). The normalization was done with a fraction of 0.2 on all data points.

For each cDNA, four values were obtained, two for one slide and two for the dye-swap. Genes with two or more missing values were removed from further analysis. Missing values were possibly due to a bad signal-to-noise ratio. A gene was considered to be differentially expressed when the mean value of the ratio was >2 or <−2 and the cDNA was identified with significance analysis of microarrays (based on SAM (Tusher et al., 2001)) with a False discovery rate <2%. Because a ratio is expressed in a log₂ scale, a ratio of >2 or <−2 corresponds to a more than four-fold up- or down-regulation, respectively.

Sequencing and Sequence Analysis

Bacterial clones containing an insert representing a differentially expressed gene were sequenced. First, a PCR was performed. One reaction of 50 μl contained: 5 μl of 10×ExTaq buffer (TaKaRa), 1 μl dNTP mixture (2.5 mM each, TaKaRa), 0.1 μl nested primer 1 (5′-TCGAGCGGCCGCCCGGGCAGGT-3′) (SEQ ID NO:_) and nested primer 2 (5′-AGCGTGGTCGCGG CCGAGGT-3′, 100 μmol/μl) (SEQ ID NO:_), 0.125 μl TaKaRa ExTaq (5 units/μl), 43.58 μl sterilized distilled water and a bacterial clone from the library. The PCR was performed using a thermocycler (Primus) programmed to conduct the following cycles: two minutes at 95° C., 40×(45 seconds at 95° C., 45 seconds at 69° C., 120 seconds at 72° C.), five minutes at 72° C. The PCR amplification products were purified using Sephadex G50 fine column filtration.

One μl of the purified PCR product was sequenced using 10 pmol of nested primer 1 and 4 μl of ABI PRISM BigDye Terminator Cycle Sequencing Ready reaction in a total volume of 10 μl. The sequence reaction consisted of two minutes at 96° C., 40×(ten seconds at 96° C., four minutes at 60° C.). Sequencing was performed on an ABI 3700 DNA sequencer. Sequence results were analyzed using SeqMan 5.00. Sequences were compared with the NCBI non-redundant and the EST Gallus Gallus database using blastn and blastx options (Altschul et al., 1997). A hit was found with the blast search when the E-value was lower than 1E-5. For unknown chicken genes, the accession number of the highest hit with the Gallus Gallus EST database is given and a description of the highest blastx hit. For known chicken genes, the accession number is given.

Quantitative LightCycler Real-Time PCR

For a reverse transcription, 200 ng RNA was incubated at 70° C. for ten minutes with random hexamers (0.5 μg, Promega). After five minutes on ice, the following was added: 5 μl 5×first strand buffer (Life Technologies), 2 μl 0.1 M DTT (Life Technologies), 1 μl Superscript RNase H— reverse transcriptase (200 Units/μl, Life Technologies), 1 μl RNAsin (40 Units/μl, Promega), 1 μl 2 mM dNTP mix (TaKaRa), water till a final volume of 20 μl. The reaction was incubated for 50 minutes at 42° C. The reaction was inactivated by heating at 70° C. for ten minutes. Generated cDNA was stored at −20° C. until use.

PCR amplification and analysis were achieved using a LightCycler instrument (Roche). For each primer combination, the PCR reaction was optimized (Stagliano et al., 2003). The primers are shown in Table 2. The reaction mixture consisted of 1 μl cDNA (1:10 diluted), 1 μl of each primer (10 μM solution), 2 μl LightCycler FastStart DNA Master SYBR Green mix, MgCl₂ in a total volume of 20 μl. All templates were amplified using the following LightCycler protocol: a pre incubation for ten minutes at 95° C.; amplification for 40 cycles: (five seconds at 95° C., ten seconds at annealing temperature, 15 seconds at 72° C.). Fluorescent data were acquired during each extension phase. After 40 cycles, a melting curve was generated by heating the sample to 95° C. followed by cooling down to 65° C. for 30 seconds and slowly heating the samples at 0.2° C./second to 96° C. while the fluorescence was measured continuously.

In each run, four standards of the gene of interest were included with appropriate dilutions of the cDNA to determine the cDNA concentration in the samples. All RT-PCRs amplified a single product as determined by melting curve analysis.

Results

Differences Between Control and MAS-Induced Chickens

All chickens inoculated with the MAS homogenate developed growth retardation, which is the main clinical feature of MAS. A significant reduction in body weight gain relative to the controls was found in the susceptible chickens, compared to the body weight gain reduction in the resistant chickens after MAS induction (data not shown). A comparison of the gene expression in the chicken intestine was made in control and MAS-induced chickens for the time points eight hours, one, three, five, seven and eleven days post-inoculation of both broiler lines. The hybridization experiments showed different numbers of up- and down-regulated genes after the MAS induction (Table 3). In general, more genes were found differentially expressed in the MAS-susceptible broiler line compared to the resistant line. At day 1 post-inoculation, most differentially expressed genes were found in both lines. The identity of the different up- and down-regulated genes is shown in Table 4. To investigate if these genes are generally induced or repressed after a MAS induction, hybridizations were repeated with samples from animal experiment 2, where the same chicken lines were used. Samples were available from days 1, 3 and 13 post-inoculation. The majority of the up- or down-regulated genes were found in both experiments (data not shown).

Differences Between MAS-Susceptible and -Resistant Broiler Lines

The results of the comparison of infected versus control chickens indicated that there are clear gene expression differences between the two chicken lines used. Therefore, samples from the two chicken lines were compared in a control situation or in an MAS-induced situation. In the control situation, no significant differences between the two broiler lines were found except at day 11. Here, 17 genes were identified that were expressed at least four-fold higher in the susceptible line at day 11, with a false discovery rate lower than 2% (Table 5). In the MAS-induced situation at day 11, these genes differed insignificantly between the two lines, most log₂ ratios of these expression differences were between −1.0 and 1.0 with only two exceptions.

For the MAS-affected situation, only significant differences between the two broiler lines were found at day 7 post-inoculation, with a false discovery rate lower than 2% and at least a four-fold expression difference. However, at days 1 and 11 post-inoculation in the MAS-affected situation, genes were identified with a false discovery rate of 2.1 and 2.2%, respectively. These genes were here also considered to be significantly different in their expression levels. An overview of the genes differing between the two lines in the MAS-induced situation is given in Table 6. All these genes lacked significant expression differences in the control situation with log₂ ratios between −1.0 and 1.0.

Confirmation of Gene Expression Differences

Array results are often influenced by each step of the complex assay, from array manufacturing to sample preparation and image analysis. Validation of expression differences is, therefore, preferably performed with an alternate method. LightCycler RT-PCR was chosen for this validation because it is quantitative, rapid and requires only small amounts of RNA.

Eight differentially expressed genes were chosen for validation. They were differentially expressed in MAS-induced chickens compared to control chickens and/or were differentially expressed between the two chicken lines. Pools of RNA were tested for all time points. For the time point with the largest differences in gene expression, five individual animals were tested in the LightCycler. In contrast to the microarray, (relative) concentrations of mRNA are measured in the LightCycler RT-PCR, while the microarray detects expression differences. Therefore, the average was taken of the LightCycler results of the individual animals and then converted to log₂ (infected/control). For all eight genes tested, the results with the pools of RNA were similar for the LightCycler and the microarray (Table 7). For seven of the eight genes tested, the differences between two groups were significant for individual animals (p<0.05). Only for gastrotropin at day 1 post-inoculation, the distribution of the results within the groups was widely spread.

Differences in gene expression in control conditions between the broiler lines were detected on day 11. This means that the gene expression levels at earlier time points are comparable in these two broiler lines in the control situation. Therefore, all differences found in the MAS-induced situation at earlier time points are due to MAS and not to other differences. The identified gene expression differences at day 11 either have a role in energy metabolism, the immune system, or they are not yet characterized. Gene expression differences at day 11 are important for the rate of recovery of the intestinal lesions, which might also influence MAS susceptibility.

TABLE 2
Sequences of used primers for LightCycler RT-PCR

| Gene | | |
|---|---|---|
| name/homology | Forward primer | Reverse primer |
| Avian nephritis | ATTGCACAGTCAACTAATTTG | AAAGTTAGCCAATTCAAAATTA |
| virus | (SEQ ID NO:_) | (SEQ ID NO:_) |
| Calbindin | CATGGATGGGAAGGAGC | GCTGCTGGCACCTAAAG |
| | (SEQ ID NO:_) | (SEQ ID NO:_) |
| Gastrotropin | TAGTCACCGAGGTGGTG | GCTTTCCTCCAGAAATCTC |
| | (SEQ ID NO:_) | (SEQ ID NO:_) |
| HES1 | TCTTCCCAGGCTGTGAG | GGTCACCAGCTTGTTCTTC |
| | (SEQ ID NO:_) | (SEQ ID NO:_) |
| Interferon-induced | CGATCATGTCTGGTGAGGC | AGCACCTTCCTCCTTTG |
| 6-16 protein | (SEQ ID NO:_) | (SEQ ID NO:_) |
| Lysozyme G | CGGCTTCAGAGAAGATTG | GTACCGTTTGTCAACCTGC |
| | (SEQ ID NO:_) | (SEQ ID NO:_) |
| Meprin | TTGCAGAATTCCATGATCTG | AGAAGGCTTGTCCTGATG |
| | (SEQ ID NO:_) | (SEQ ID NO:_) |
| Pyrin | CCTGCACTGACCCTTG | GTGGCTCAGGGTCTTTC |
| | (SEQ ID NO:_) | (SEQ ID NO:_) |

TABLE 3
Number of differentially expressed genes in Malabsorption-affected
chickens at different time points in different broiler lines

| | | | | | |
|---|---|---|---|---|---|
| 8 hours | | | | | day 11 |
| pi | day 1 pi | day 3 pi | day 5 pi | day 7 pi | pi |

Number of induced genes

| | | | | | | |
|---|---|---|---|---|---|---|
| Susceptible line | 7 | 31 | 14 | 17 | 3 | 6 |
| Resistant line | 0 | 38 | 11 | 0 | 2 | 0 |

Number of repressed genes

| | | | | | | |
|---|---|---|---|---|---|---|
| Susceptible line | 0 | 9 | 0 | 16 | 16 | 2 |
| Resistant line | 0 | 7 | 3 | 0 | 2 | 0 |

TABLE 4
Genes and ESTs four-fold up- or down-regulated after a MAS
induction

| chicken gene | description | Susceptible line | Resistant line |
|---|---|---|---|
| U73654.1 | alcohol dehydrogenase | d1 | d1 |
| AF008592.1 | inhibitor of apoptosis protein1 | d1 | |
| U00147 | filamin | d1 | |
| X52392.1 | mitochondrial genome | d1 u5, 11 | |
| M31143.1 | calbindin | d1, 5, 7, 11 | u1, d7 |
| AJ236903.1 | SGLT-1 | d5 | |
| AJ250337.1 | cytochrome P450 | d5, 7 | d1 |
| M18421.1 | apolipoprotein B | d5, 7 | |
| M18746.1 | apolipoprotein AI | d5, 7 | |
| AF173612.1 | 18S rRNA | u8hr | u3, 7 |
| AF469049.1 | caspase 6 | u1 | u1 |
| U50339.1 | galectin-3 | u1 | u1 |
| AJ289779.1 | angiopoietin 2C | u1, 3, 5 | d1 |
| L34554.1 | stem cell antigen 2 | u1, 5 | u1, 3 |
| D26311.1 | unknown protein | u11 | |
| AJ009799.1 | ABC transporter protein | u3 | d1 |
| M10946.1 | aldolase B | u3 | u1, 3 |
| AF059262.1 | cytidine deaminase | u5 | u1 |
| AJ307060.2 | ovocalyxin-32 | u5 | |
| M27260.1 | 78 kDa glucose regulated protein | u5 | |
| AY138247.1 | p15INK4b tumor suppressor | | d7 |
| AJ006405.1 | glutathion-dependent prostaglandin D | | u1 |
| | synthase | | |
| chicken EST | homology | Susceptible line | Resistant line |
| BU123833 | annexin A13 | d1 | |
| CD727681 | pyrin | d1 | |
| BU420110 | | d1, 7 | |
| BU124420 | liver-expressed antibacterial peptide 2 | d5 | |
| BU217169 | sucrase-isomaltase | d5 | d1, 3 |
| BU292533 | tubulointerstitial nephritis antigen-related | d5 | |
| | protein | | |
| CD726841 | zonadhesin | d5 | |
| BU123839 | | d5 | d1, 3 |
| BU124534 | meprin | d5, 7 | |
| BU262937 | angiotensin I converting enzyme | d5, 7 | |
| BU288276 | mucin-2 | d5, 7 | |
| BU480611 | | d5, 7 | u1 |
| BU124511 | Na+/glucose cotransporter | d7 | |
| BU268030 | | d7 | |
| BU464138 | | d7 | |
| BU122834 | pyrophosphatase/phosphodiesterase | u8hr | d1 |
| BU122899 | fatty acyl CoA hydrolase | u8hr, 1 | u1 |
| BU467833 | interferon-induced 6-16 protein | u8hr, 1, 3, 5 d7 | u1, 3 |
| — | avian nephritis virus | u8hr, 1, 3, 5, 7 | u1, 3 d7 |
| | | d11 | |
| BU138064 | retionic acid and interferon inducible 58 kDa | u8hr, 1, 5 | u1, 3 |
| | protein | | |
| BX258371 | gastrotropin | u8hr, 5 d1 | d1 |
| AI982261 | ubiquitin-specific proteinase ISG43 | u1 | u1 |
| BG712944 | aminopeptidase | u1 | |
| BU125579 | cathepsin S | u1 | |
| BU233187 | zinc-binding protein | u1 | u1 |
| BU240951 | | u1 | u1 |
| BU255435 | beta V spectrin | u1 | u1 |
| BU397837 | | u1 | |
| BU492784 | putative cell surface protein | u1 | u1 |
| BX273124 | phosphofructokinase P | u1 | |
| BU249257 | unnamed protein product | u1 | u1 |
| — | | u1 | u1 |
| BU296697 | IFABP | u1, d5, 7 | u1 |
| BU302098 | C1 channel Ca activated | u1, d7 | u1 |
| BU410582 | HES1 | u1, 11 | u1, 7 |
| BU124153 | Ca activated C1 channel 2 | u1, 11 d5, 7 | u1 |
| AJ452523 | mucin-like | u1, 3 | u1 |
| BU118300 | hensin | u1, 3 | u1 |
| — | lymphocyte antigen | u1, 3 | u1 |
| CD727020 | interferon induced membrane protein | u1, 3, 5 | u1, 3 |
| BU401950 | lysozyme G | u1, 3, 5, 7 | u1, 3 |
| BU452240 | 14 kDa transmembrane protein | u1, 3, 5, 7 | u1, 3 |
| BU244292 | transmembrane protein | u1, 5 | u1 |
| BX271857 | No homology | u1, 5 | u1, 3 |
| — | | u11 | |
| — | immunoresponsive gene 1 | u11 | |
| BU305240 | | u3 | |
| BU130996 | anterior gradient 2 | u3, 5 | |
| BU378220 | | u5 | u1 |

d = down-regulated at the indicated time point(s).
u = up-regulated at the indicated time point(s), 8 hours, 1, 3, 5, 7 or 11 days post-inoculation.
— = no EST in the database (August 2003)

TABLE 5
Genes expressed higher in the susceptible line compared to the
resistant line in control situation at day 11

| | | log₂ ratio in | log₂ ratio in MAS |
|---|---|---|---|
| EST | Chicken gene/homology | control situation | induced situation |
| BU123839 | No homology | 3.7 | 0.3 |
| BU118300 | hensin | 3.7 | 1.7 |
| BX271857 | No homology | 3.5 | 0.2 |
| — | Avian nephritis virus | 3.3 | −0.5 |
| | Mitochondrial genome* | 2.8 | 0.2 |
| | cytochrome C oxidase subunit 1* | 2.5 | 0.1 |
| BU123664. | No homology | 2.3 | −0.0 |
| BU401950 | lysozyme G | 2.3 | 1.1 |
| BU467833 | interferon-induced 6-16 protein | 2.3 | 0.2 |
| | plasma membrane calcium pump* | 2.2 | −0.1 |
| BU124318 | immune associated nucleotide protein | 2.2 | −0.1 |
| | Stem cell antigen 2* | 2.2 | 0.0 |
| — | lymphocyte antigen | 2.2 | 0.1 |
| | cytochrome C oxidase subunit III* | 2.1 | 0.1 |
| BX257981 | No homology | 2.1 | 0.3 |
| — | No homology | 2.0 | 0.6 |
| — | No homology | 2.0 | 0.9 |

*= chicken gene
— = no EST in the database (August 2003)

TABLE 6
Genes and ESTs significantly differentially expressed in one of the
broiler lines after a MAS induction

| | | | | ratio | ratio |
|---|---|---|---|---|---|
| EST | Chicken gene/homology | day | line¹ | MAS² | control³ |
| | SGLT-1* | 1 | S | 2.2 | −0.0 |
| BU233187 | zinc-binding protein | 1 | R | 2.2 | 0.1 |
| AJ295030 | aldo-ketoreductase | 1 | R | 2.3 | 0.8 |
| BU307467 | retinol-binding protein | 1 | R | 2.4 | −0.5 |
| BX258371 | gastrotropin | 1 | R | 2.6 | 0.7 |
| CD727681 | pyrin | 1 | R | 3.2 | −0.3 |
| — | Avian nephritis virus | 7 | S | 3.2 | −0.2 |
| BU401950 | lysozyme G | 7 | S | 2.7 | 0.7 |
| BU296697 | IFABP | 7 | R | 2.2 | 0.3 |
| BU268030 | no homology | 7 | R | 2.2 | 0.1 |
| | cytochrome P450* | 7 | R | 2.5 | −0.2 |
| | glutathion-dependent | 7 | R | 2.5 | 0.9 |
| | prostaglandin D | | | | |
| | synthase* | | | | |
| BU124534 | meprin | 7 | R | 2.7 | −0.6 |
| | Calbindin* | 7/11 | R | 2.8/2.1 | −0.4/−0.4 |
| | cytidine deaminase* | 11 | S | 2.0 | 0.3 |

*= chicken gene
— = no EST in the database (August 2003)
¹Broiler line with higher expression after MAS induction
²log₂ ratio in MAS-induced situation
³log₂ ratio in control situation

TABLE 7
Results of LightCycler RT-PCR for eight genes compared with the
microarray results

| | | array | LightCycler | | LightCycler |
|---|---|---|---|---|---|
| | | susceptible | susceptible | array resistant | resistant |
| gene name | day | infected/control | infected/control | infected/control | infected/control |
| anv | 1 | 2.8 | NA* | 1.9 | NA* |
| calbindin | 7 | −3.5 | −2.7 | −1.2 | −3.2 |
| gastrotropin | 1 | −2.7 | −2.3 | −2.3 | −2.6 |
| HES1 | 1 | 1.9 | 2.9 | 1.7 | 2.4 |
| interferon-induced | 1 | 2.4 | 3.0 | 4.1 | 3.1 |
| 6-16 protein | | | | | |
| lysozyme G | 1 | 3.4 | 11.2 | 3.8 | 13.4 |
| meprin | 7 | −3.3 | −3.4 | −0.6 | −1.3 |
| pyrin | 1 | −4.2 | −2.4 | 0.4 | 0.4 |

*All the control animals remain negative in the LightCycler experiment, therefore no ratio could be calculated.

### EXAMPLE 2

### Small Intestinal Segment Perfusion Test (SISP) in Pigs

We have developed a porcine small intestinal microarray based on cDNA from jejunal mucosal scrapings. Material from two developmental distinct stages was used in order to assure a reasonable representation of mucosal genes. Pig muscle cDNA was used for subtraction and normalization. The microarray consists of 3468 spotted cDNAs in quadruplicate. Comparison of the two sources revealed a differential expression in at least 300 genes. Furthermore, we report the early response of pig small intestine jejunal mucosa to infection with enterotoxic E. Coli (ETEC) using the small intestinal segment perfusion (SISP) technique. A response pattern was found in which a marker for innate defense dominated. Further analysis of these response patterns will contribute to a better understanding of enteric health and disease in pigs. The great similarity between pig and human indicate results to be applicable for both agricultural and human biomedical purposes.

Materials and Methods

Pigs

For the construction of the microarray, pigs were used (Dalland synthetic line, with a large White/Pietrain background) from the pig farm from the Animal Sciences Group. Pigs used for the SISP technique were purchased from a commercial piggery and were cross-bred Yorkshire×(Large White×Landrace).

All animal studies were approved by the local Animal Ethics Commission in accordance with the Dutch Law on Animal Experimentation.

Material for Microarray

Four pigs, 12 weeks old, two males, two females, from four different litters, feed and water ad lib, without clinical symptoms, no diarrhea, normal habitus and body weight, were selected by the investigator and transported to the necropsy room. Furthermore, four piglets, four weeks old, two males, two females, from two different litters, clinically healthy, were weaned and transported to the experimental unit. Piglets were fasted for two days, receiving water ad lib, followed by transport to necropsy room. In the necropsy room, animals were killed by intravenous barbiturate overdose, and the intestines were taken out. Jejunum was opened, rinsed with cold saline, and the mucosa of 10 cm of jejunum were scraped off with a glass slide. Mucosal scrapings were snap frozen in liquid nitrogen and kept at −70° C. until further use. Adjacent parts of the jejunum were fixed in 4% formaldehyde and used for histology. Villus and crypt dimensions were determined on hematoxylin eosin stained 5 nm tissue sections according to Nabuurs et al., 1993b.

Determination of F4 Receptor Status Previous to the SISP-Technique

Under inhalation anesthesia, biopsies were taken from the proximal duodenum, using a fiberscope (Olympus GIF XP10, Hamburg, Germany) under endoscopic guidance. A minimum of four forceps biopsies were taken using a Biopsy forceps channel diameter 2 mm (Olympus Hamburg, Germany). Biopsies were stored in 0.5 ml PBS at 4° C. F4 receptor status was determined using the brush border adhesion assay modified after Sellwood et al., 1975. Briefly, biopsies were homogenized using an Ultrasonic Branson 200 sonifier, the resulting brush border membranes were incubated with 0.5 ml 10⁹ CFU/ml E. coli F4 (CVI-1000 E. coli O149K91 strain (Nabuurs et al., 1993a)) in PBS containing 0.5% mannose and incubated at room temperature for 45 to 60 minutes. Adhesion was judged by phase contrast microscope. Furthermore, E. coli bacteria lacking F4 fimbriae (CVI-1084) (van Zijderveld et al., 1998) were used to corroborate the specificity of F4-mediated adhesion. After a SISP experiment, F4-receptor status was confirmed using larger amounts of intestinal scrapings.

Small Intestinal Segment Perfusion Test (SISP)

The SISP was performed essentially as described by Nabuurs et al., 1993a; Kiers et al., 2001). Briefly, pigs (9 to 10 kg) were sedated with 0.1 ml azaperone (Stressnil) per kg bodyweight. After 15 minutes, inhalation anesthesia was performed with a gas mixture of 39% oxygen, 58% nitrous oxide and an initial 3% isoflurane; after ten minutes, 2% halothane. The abdominal cavity was opened and about 40 cm caudal from the ligament of Treitz, the first pair of segments of 20 cm length was prepared by inserting a small inlet tube in the cranial site of a segment and by inserting a wide outlet tube into the caudal site of a segment at 10% of the total length of the small intestine. Four other pairs of segments were prepared similarly at 25%, 50%, 75%, and 95% in the small intestine. While preparing the segments, a swab was taken and plated on sheep blood agar plates, which were incubated for 24 hours at 37° C., to check for the presence of endogenous hemolytic E. coli. Perfusion was performed manually with syringes attached to the cranial tubes, 2 ml every 15 minutes. Effluent was collected in 100 ml bottles. Segments were perfused for eight hours with 64 ml of perfusion fluid (9 g NaCl, 1 g Bacto casaminoacids (Difco), and 1 g glucose per liter distilled water). From a pair of segments, one was before-perfusion infected with 5 ml of 10⁹/ml PBS enterotoxic E. coli F4 (CVI-1000 E. coli O149K91 strain Nabuurs et al., 1993a), the other was mock infected with vehicle only. After perfusion, fluid remaining in a segment was also collected and the pigs were euthanized by barbiturate overdose. The surface area of each segment was measured. Net absorption was defined as the difference between inflow and outflow in ml/cm². Mucosal scrapings were taken for genomic analysis from four animals. From each animal, a segment was taken, one with and one without E. coli, and frozen at −70° C. Pairs used were located around 25% of small intestine, in the anterior jejunum. Furthermore, mucosal scrapings were taken for conformation of the F4-receptor status as described above.

Isolation of Total RNA

Approximately 1 gram of frozen tissue (mucosal scrapings) collected from four- and twelve-week-old pigs or from SISP segments (see above), was homogenized directly in 10 ml TRIzol reagent (GibcoBRL). After homogenization, insoluble material was removed from the homogenate by centrifugation at 12,000×g for ten minutes at 4° C. Further extraction of RNA from these homogenates was performed according to instructions of the manufacturer of TRIzol reagent. The crude RNA pellet obtained from this isolation procedure was dissolved in 1 ml RNase-free water and precipitated with 0.25 ml of isopropanol and 0.25 ml of 0.8 M sodium citrate/1.2 M NaCl to remove proteoglycan and polysaccharide contamination. After centrifugation at 12,000×g for ten minutes at room temperature, RNA pellets were washed with 75% (v/v) ethanol and dissolved in RNase-free water. Subsequently, the RNA was treated with DNase, extracted once with phenol-chloroform, and precipitated with ethanol. RNA pellets were washed with 75% (v/v) ethanol, dissolved in RNAse-free water, and stored at −70° C. until further use. The integrity of the RNA was checked by analyzing 0.5 μg on a 1% (w/v) agarose gel.

Construction and Hybridizing of the Microarray

Equal amounts of total RNA extracted from each four-week-old pig (4 wkM) were pooled, and a similar pool was prepared of RNAs isolated from the four twelve-week-old pigs (12 wkS). One microgram of pooled RNA was used to construct a cDNA library of expressed sequence tags (ESTs) using the SMART™ PCR cDNA synthesis KIT (Clontech). To remove redundant cDNAs, the cDNA generated from the twelve-week-old pigs was subtracted with a portion of homologue cDNA (normalized) and the cDNA of the four-week-old pigs was subtracted with pig muscle cDNA (using the PCR-select™ subtraction kit; Clontech). EST fragments were cloned in a pCR4-TOPO vector using DH5α-T1^{R} cells (Invitrogen). Individual library clones were picked and grown in M96 wells containing LB plus 10% (v/v) glycerol and 50 μg/ml ampicillin, and M96 plates were stored at −70° C. A total of 672 EST fragments from the muscle-subtracted library (four-week-old pigs) and 2400 from the normalized library (twelve-week-old pigs) were amplified by PCR and spotted in quadruplicate on microarray slides as described (van Hemert et al., 2003).

Before hybridization, the microarray was pre-hybridized in 5% SSC, 0.1% SDS and 1% BSA at 42° C. for 30 minutes. To label the RNA, MICROMAX TSA labeling and detection kit (PerkinElmer) was used. The TSA probe labeling and array hybridization were performed as described in the instruction manual with minor modifications. Biotin- and fluorescein-labeled cDNAs were generated from 1 or 2 μg of total RNA isolated from the SISP segments per reaction. The cDNA synthesis time was increased to three hours at 42° C., as suggested (Karsten et al., 2002). Post-hybridization washes were performed according to the manufacturer's recommendations. Hybridizations were repeated with the fluorophores reversed (dye swap). After signal amplification, the microarrays were dried and scanned in a Packard Bioscience BioChip Technologies apparatus (PerkinElmer). The image was processed (Scanarray™-express software, PerkinElmer) and spots were located and integrated with the spotting file of the robot used for spotting. Reports were created of total spot information and spot intensity ratio for subsequent data analyses.

Analysis of the Microarray Data

After background correction, the data presented in an M/A plot were M=log₂R/G and A=log₂√(R×G) (Dudoit et al., 2002). An intensity-dependent normalization was performed using the lowest function in the statistical software package R (Yang et al., 2002). The normalization was done with a fraction of 0.2 on all data points.

For each EST, six values were obtained, three for one slide and three for the dye-swap. Genes with three or more missing values were removed from further analysis. Missing values were possible due to a bad (local) signal-to-noise ratio. A gene was considered to be differentially expressed when the mean value of the ratio was >2 or <−2 and the cDNA was identified with significance analysis of microarrays (based on SAM (Tusher et al., 2001)) with a False discovery rate <2%. Because a ratio is expressed in a log₂ scale, a ratio of >2 or <−2 corresponds to a more than four-fold up- or down-regulation, respectively.

Sequencing and Sequence Analysis

The inserts (ESTs) of the bacterial clones that hybridized differentially were amplified by PCR using primers complementary to the multiple cloning site of the pCR4-TOPO cloning vector, purified and sequenced using nested primer 1 (5′-TCGAGCGGCCGCCCGGGCAGGT-3′) (SEQ ID NO:_) or nested primer 2R (5′-AGCGTGGTCGCGGC CGAGGT-3′) (SEQ ID NO:_), both complementary to the sequence of the adaptors 1 and 2R ligated to termini of the EST fragments (see manual PCR-select™ subtraction kit, Clontech). Sequence reactions were performed using the ABI PRISM BigDye Terminator Cycle Sequencing kit and reactions were analyzed on an ABI 3700 DNA sequencer. Sequence results were analyzed using SeqMan 5.00 and compared with the NCBI non-redundant and the porcine and human EST databases (TIGR) using blastn and blastx options (Altschul et al., 1997).

Northern Blot Analysis

Equal amounts of total RNA (5 or 10 μg) were separated on a denaturating 1% (w/v) agarose gel and blotted on Hybond-N membranes (Amersham) as described (Sambrook et al., 1989). Plasmid DNA was isolated from EST library clones that hybridized differentially on the microarray slides.

After restriction enzyme digestion of a DNA fragment, homologues to the coding sequence of the gene that scored the lowest E-value in the blastx analysis (see above) was purified from gel. Fifty nanograms of DNA fragment was labeled with 50 μCi of [α-³²P]-dCTP (3000 Ci/mmol) using the random primer kit (Roche) and used as probe to hybridize RNA blots. Blots were hybridized using probes with a specific activity of approximately 10⁸ cpm/μg DNA in a solution containing 40% (v/v) Formamide and 5×SSPE overnight at 42° C. (Sambrook et al., 1989). The blots were scanned using a Strom phosphor-imager (Molecular Dynamics, Sunnyvale, Calif.) and the pixel intensity of each individual band was determined using Image-Quant® software (Molecular Dynamics). Differential expression was calculated as the ratio of pixel intensity of E. coli infected over mock infected.

Results

Construction of the Pig Intestinal cDNA Microarray

The development of the pig intestinal cDNA microarray was based on total RNA extracted from two developmentally distinct types of jejunal mucosa. One source was a mucosal pool from four animals of four weeks old that were just weaned (4 wkM). The other source was a pool of four twelve-week-old pigs that were fed conventionally (12 wkS). Histologically, 4 wkM was characterized by high villi and a high villus/crypt ratio; 12 wkS showed shorter villi and a lower villus/crypt ratio (Table 8). Isolated RNA showed no degradation on agarose gel analysis. Pooled RNA was used to construct a cDNA library of expressed sequence tags (ESTs). To reduce redundant cDNA, the cDNA generated from 12 wkS was subtracted with a portion of homologue cDNA (normalized) and the cDNA of 4 wkM was subtracted with pig muscle cDNA. Sequencing of 100 randomly picked clones revealed that approximately 5% had no insert, 90% represented clones with unique sequences, and 5% was present in two or more fold. This degree of redundancy was considered acceptable. A total of 672 EST fragments from 4 wkM and 2256 from 12 wkS were spotted in quadruplicate on microarray slides. One hundred twenty-eight annotated EST fragments selected from the Marc1 and Marc2 EST libraries were added, and eleven other known EST were from our own laboratory, some of those in duplicate. Three hundred eighty-four controls for hybridization and labeling were spotted too, yielding a microarray consisting of 3468 spotted cDNAs in quadruplicate.

Assessment of the Degree of Variation Between the Two Developmental Stages

To evaluate the degree of variation between 4 wkM and 12 wkS, both were analyzed on the microarray. A gene was considered to be differentially expressed when the mean value of the ratio was larger then four. Using this cut-off, 300 spots with differential expression were identified, 220 were up-regulated in 4 wkM, and 80 were up-regulated in 12 wkS. Fifty up-regulated spots from each were sequenced and functionally clustered based on (tentative) function (Table 9).

Analysis of a Differential Expression in the Mucosa of Normal Versus Enteropathogenic E. Coli-Infected Small Intestinal Loops

To examine the utility of the microarray in detecting meaningful differences in gene expression, we compared mucosal cDNA from normal uninfected with enteropathogenic E. coli-infected small intestinal loops using the SISP technique. The latter is a technique that we frequently use for the testing of functional foods (e.g., Kiers et al., 2001). The technique requires piglets expressing the receptor for the F4 fimbrium, expressed by enteropathogenic E. coli, which is determined beforehand by peroral biopsy of the duodenum. In a typical experiment, in each of four F4 receptor-positive piglets, ten small intestinal loops are made. In each piglet, a mock-infected loop and an E. coli-infected loop is present. The loops are perfused during eight hours, and net absorption is calculated. From one of our experiments, mucosal scrapings were taken from the mock-infected and the infected loops from each of the four pigs. The average (±SD) net absorption of the four mock-infected segments was 571±299 microL/cm², of the E. coli-infected segments −171±189 microL/cm², which means that there was average net excretion in enterotoxic E. coli-infected loops. Cultures of swabs taken from the intestinal loops before the experiment confirmed the absence of hemolytic E. coli.

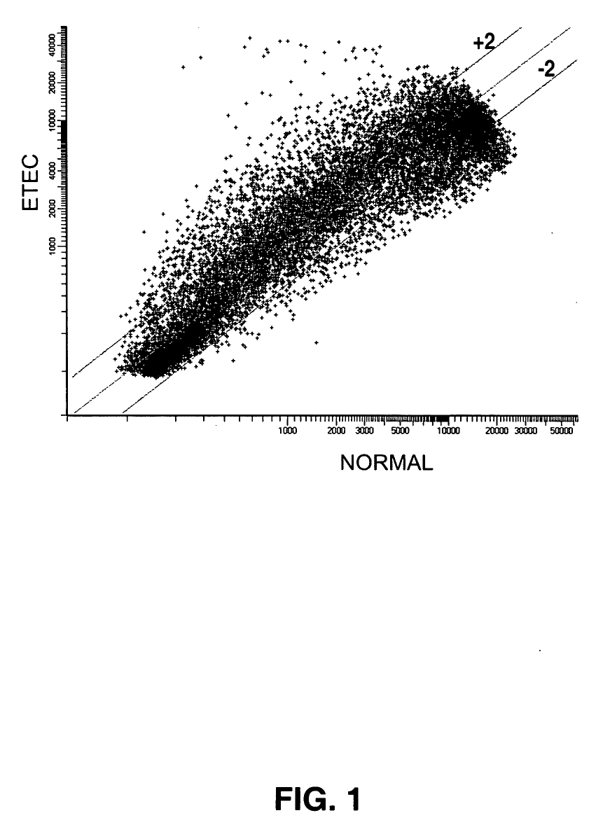

Dual-color hybridization was performed on two slides. In FIG. 1, a typical example (animal 6) of the expression of each spot is plotted. Most points cluster around the middle line and within the limits set for differential expression (+2, and −2), indicating similar levels of expression in both tissues. About 100 spots did fall significantly, either above or under the middle line, indicating differential expression.

Comparing within animals (isogenic), E. coli versus mock-infected, in animals 6, 7, and 8, on average 102 spots were found to be differentially expressed, 75±4 up and 28±4 down (±SD). In animal 5, differential expression was found in close to 500 spots, of which 300 up and 200 down. Since animal 5 appeared to be quite different from the other animals, only animals 6, 7 and 8 were used for further analysis of the average differential expression. The latter animals had 24 differentially expressed spots in common, of which 16 up- and 8 down-regulated. Sequencing of these spots revealed these represented 15 different genes, of which 10 up- and 5 down-regulated. The most markedly (>30 times) elevated expression in these three animals is of a gene identified as pancreatitis-associated protein (PAP).

Validation of the Microarray by Northern Blot

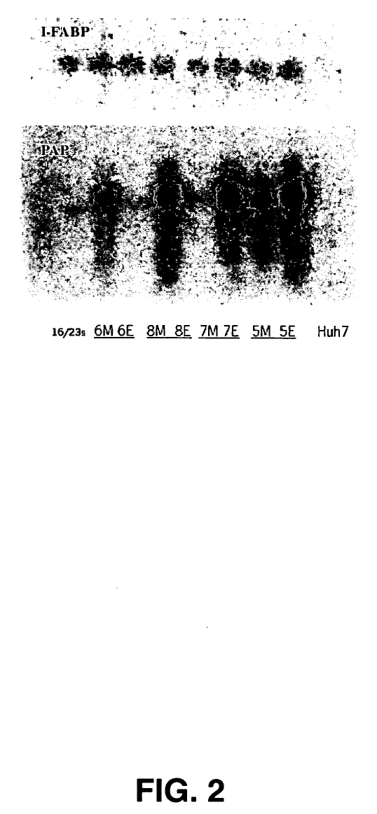

Validation of expression differences found with microarray with an alternate method is essential. In our pig model, sufficient material is available to use analysis by Northern blot (NB). Concerning I-FABP, comparison of expression between microarray and NB revealed no essential differences (FIG. 2 and Table 10). Concerning PAP, in three out of four segments pairs (5, 6, and 7), similar values were obtained in by both microarray and NB analysis. In segment pair 8, the microarray gave a four-fold overestimation of PAP-expression as established by NB. No PAP-expression was found in mock-infected segments except in segment pair 5.

In order to obtain a relatively wide range of genes, two different sources of mucosa were used that are known to vary in differentiation (Nabuurs et al., 1993b; van Dijk et al., 2002), and immunological maturation. The first group consisted of young four-week-old animals, which were taken just after weaning (4 wkM). The mucosa of these animals is morphologically characterized by large villi, a high villus crypt ratio, and their epithelial metabolism is geared towards the digestion of milk. The other group consisted of twelve-week-old conventionally solid fed (12 wkS) animals, with a more mature mucosa with short villi and a lower villus crypt (V/C) ratio.

Histological analysis showed that in both groups, villus and crypt dimensions and V/C ratio were consistent with the literature (Nabuurs et al., 1993b; van Dijk et al., 2002). Four animals per group were used, with equal representation of both sexes. Jejunal mucosa was harvested by scraping and total RNA pooled per group was used to generate two independent EST (cDNA) libraries. The cDNA obtained from 4 wkM was subtracted with muscle cDNA, and that of 12 wkS was subtracted with homologous cDNA (normalization). Sequencing of 100 random clones revealed the degree of redundancy. Redundancy on the one hand reduces the amount of genes detected; on the other hand, it can reduce the problem of saturation by highly prevalent mRNAs (Hsiao et al., 2002). Close to 3000 unknown ESTs, amplified from both libraries, were spotted on the microarray. Furthermore, 140 annotated EST fragments selected from the Marc1 and Marc2 EST libraries (Fahrenkrug et al., 2002), and controls were added.

One of the problems anticipated is that differences found between samples would rather represent differences in cell type distribution than in cellular responses. We, therefore, wanted to include a specific marker for the relative amount of epithelium. A suitable candidate was intestinal fatty acid binding protein (1-FABP), a protein exclusively expressed in the small intestine, with the highest tissue content in the jejunum (Pelsers et al., 2003).

Ideally, I-FABP mRNA should be constitutive; this is, however, not entirely clear (Glatz and van der Vusse, 1996). Nevertheless, I-FABP mRNA has been described in rats with damaged and regenerating epithelium as the least affected of a series of enterocyte-specific markers (Verburg et al., 2002). Earlier, we have demonstrated I-FABP mRNA and protein to be present in pig jejunum (Niewold et al., 2004). Therefore, I-FABP cDNA was added to the microarray as an additional control and possible standard for epithelial content.

The strategy followed to test and validate the constructed microarray was as follows. First, a cDNA from 4 wkM was tested against 12 wkS to get an estimate of the degree of variation between the two sources used for the microarray. Second, to examine the utility of the microarray in detecting meaningful differences in gene expression, we compared mucosal cDNA from normal uninfected with enteropathogenic E. coli-infected small intestinal loops. Selected genes were sequenced. Third, to validate the microarray, we compared the expression level of two selected genes as established by microarray with expression levels on Northern blot.

First, a comparison was made to establish variation between 4 wkM and 12 wkS. A gene was considered to be differentially expressed when the mean value of the ratio was larger than four. Using this cut-off, 300 spots with differential expression were identified, 220 were up-regulated in 4 wkM, and 80 were up-regulated in 12 wkS. Despite the present redundancy, this shows that there are relatively large differences in the number of genes expressed between the two developmental stages. Sequencing of differentially expressed spots revealed genes that were clustered on (tentative) function. Differences found concerned metabolism and immune-associated expression.

Second, a comparison was made to establish differential expression or normal versus enteropathogenic E. coli-infected small intestinal loop using the SISP technique. In this technique, differences over eight hours represent the acute response. Functionally, the intestinal loops showed an average normal fluid absorption in mock-infected segments and an expected average net fluid excretion in enterotoxic E. coli-infected counterparts. Comparing within animals (isogenic), E. Coli versus mock-infected, in animals 6, 7, and 8, a remarkably homogeneous result was obtained. On average, 102 spots were found to be differentially expressed, of which three-quarters up and one-quarter down. Animal 5 appeared to be aberrant in the number of differentially expressed genes (500) in the microarray. Other analysis confirmed its exceptional characteristics (see below). Animals 6, 7 and 8 had 24 differentially expressed spots in common, representing 15 different genes, of which ten up- and five down-regulated.

As expected, I-FABP expression was in all four segments below the cut-off, showing very little variation, if any. Since PAP and I-FABP genes were extremes in terms of expression differences, it was decided to use these two genes to validate with Northern blot.

Third, since array results are influenced by each step of the complex assay, validation of expression differences with an alternate method is essential. Two different methods are available: RT-PCR and Northern blot (NB). Usually, RT-PCR is chosen over Northern blot because quantities available are limiting. However, Northern blot is often superior to RT-PCR, since RT-PCR results are known to be influenced by several factors, such as the purity and integrity of the RNA, and the amplification scheme used in the RT-reaction (Chuaqui et al., 2002). In our pig model, sufficient material is available and NB was used. Concerning I-FABP, comparison of expression between microarray and NB revealed no essential differences (Table 10). Using NB, the variation (as SD) on the average value of I-FABP expression in the four segments was found to be considerably less than on those obtained by microarray (1.3±0.4 and 1.2±0.7, respectively).

TABLE 8
Histological characterization of the two different mucosas used for
construction of the microarray.

| | 4 wkM | 12 wkS |
|---|---|---|
| Villus height (μm ± SD) | 939 ± 104 | 437 ± 43 |
| Crypt depth (μm ± SD) | 135 ± 13 | 108 ± 4 |
| Villus/Crypt ratio | 6.9 ± 1.4 | 4.0 ± 0.3 |

TABLE 9
Functional Clustering of 50 genes differentially expressed in 4 wkM vs. 12 wkS.

| Nr. | | | Blast(n)/nr database | | WU-BLAST 2.0/TIGR | | (tentative) |
|---|---|---|---|---|---|---|---|
| (n) | M | | Gene name | E-value | T(H)C number | E-value | function |
| | | higher in 4 wk | | | | | |
| | | acc. number | | | | | |
| 1 (3) | 3.31 | gb\|AY208121.1\| | Sus scrofa myostatin gene, complete | e−175 | | | differen- |
| | | | cds | | | | tiation |
| 2 (3) | 3.09 | gi\|178817\| | Human apolipoprotein B-100 mRNA, | 0 | | | metabolism |
| | | | complete cds | | | | |
| 3 (2) | 3.04 | emb\|AJ504726.1 | Sus scrofa mRNA for | 2e−33 | | | metabolism |
| | | | methylmalonyl-CoA mutase | | | | |
| 4 (2) | 3.03 | emb\|AJ427478.1 | Sus scrofa ASIP gene for agouti | e−111 | | | differen- |
| | | | signaling protein | | | | tiation |
| 5 | 2.89 | emb\|AJ007302.1\| | Sus scrofa triadin gene | 1e−31 | pig\|BI405108 | 1.90E−47 | metabolism |
| 6 | 2.87 | gb\|AC097351.2\| | Sus scrofa clone RP44-368D24, | e−109 | | | unknown |
| | | | complete sequence | | | | |
| 7 | 2.87 | gb\|AC096884.2\| | Sus scrofa clone RP44-519O7, | 4e−13 | | | unknown |
| | | | complete sequence | | | | |
| 8 (3) | 2.84 | emb\|Y00705.1 | Human pancreatic secretory trypsin | 1e−22 | | | metabolism |
| | | | inhibitor (PSTI) mRNA. | | | | |
| 9 | 2.82 | emb\|AJ251829.1 | Sus scrofa MHC class I SLA genomic | 2e−45 | | | immune |
| | | | region haplotype H01 | | | | |
| 10 | 2.81 | AY116646 | Human polymerase (DNA directed), | 5.00E−73 | | | differen- |
| | | | delta 2, regulatory subunit | | | | tiation |
| 11 | 2.79 | emb\|X02747.1 | Human mRNA for aldolase B | e−165 | | | metabolism |
| 12 | 2.71 | ref\|NM_021133.2 | Homo sapiens ribonuclease L | 2e−45 | pig\|TC127834 | 4.10E−94 | immune |
| 13 | 2.71 | gb\|AF159246.1 | Bos taurus calpastatin mRNA | 1e−27 | pig\|TC117236 | 4.40E−56 | metabolism |
| 14 | 2.67 | gi\|46195796 | hypothetical protein LOC51321 | 2e−33 | pig\|TC91804 | 8.00E−104 | unknown |
| 15 | 2.67 | gi\|31874709 | Homo sapiens mRNA; cDNA | 2E−57 | pig\|TC104397 | 1.00E−89 | unknown |
| | | | DKFZp686B0790 | | | | |
| 16 | 2.67 | emb\|AL606724.17 | Mouse DNA sequence from clone | 1e−19 | | | unknown |
| | | | RP23-285D3 | | | | |
| 17 | 2.65 | gb\|U28757.1 | Sus scrofa lysozyme gene, complete | 4e−08 | pig\|BI345301 | 6.10E−25 | immune |
| | | | cds | | | | |
| 18 | 2.65 | gi\|509402\| | S. scrofa BAT1 gene | 6e−09 | pig\|BG895850 | 7.90E−18 | immune |
| 19 | 2.65 | gi\|23274203 | N-acetylgalactosaminyltransferase | 0 | | | metabolism |
| | | | (GalNAc-T) (GALGT) mRNA | | | | |
| 20 | 2.63 | gb\|U65590.1 | Homo sapiens IL-1 receptor antagonist | 7e−11 | human\| | 6.90E−25 | immune |
| | | | IL-1Ra gene | | THC1808787 | | |
| 21 | 2.62 | gb\|AF045016.1 | Canis familiaris multidrug resistance | e−111 | | | immune |
| | | | p-glycoprotein mRNA | | | | |
| 22 | 2.62 | ref\|NM_006418.3\| | Homo sapiens GW112 mRNA | 2e−05 | pig\|TC127249 | 3.20E−62 | unknown |
| 23 | 2.61 | emb\|AJ251914.1 | Sus scrofa MHC class I SLA gene | 1e−58 | | | immune |
| 24 | 2.60 | emb\|AL117672.5 | Human chromosome 14 DNA sequence | 1e−40 | human\| | 2.40E−41 | unknown |
| | | | BAC R-142C1 | | BX499816 | | |
| 25 | 2.59 | gb\|AC136964.2 | Sus scrofa domestica clone | 8e−13 | pig\|AU296464 | 2.90E−24 | unknown |
| | | | RP44-154L9. | | | | |
| 26 | 2.56 | gb\|AF282890.1\| | Sus scrofa glycoprotein GPIIIa (CD61) | 7e−39 | | | immune |
| | | | mRNA | | | | |
| 27 | 2.55 | gb\|AC092497.2\| | Sus scrofa clone RP44-30C22, | e−148 | | | unknown |
| | | | complete sequence | | | | |
| 28 | 2.49 | ref\|XM_097433.3\| | Homo sapiens hypothetical | 3e−75 | pig\|TC120374 | 4.50E−128 | unknown |
| | | | LOC148280 mRNA. | | | | |
| 29 | 2.49 | emb\|AL035683.9 | Human DNA sequence from clone | 1e−08 | pig\|TC103746 | 7.20E−72 | unknown |
| | | | RP5-1063B2 | | | | |
| 30 | 2.26 | gi\|9857226 | Sus scrofa ribophorin I | e−105 | | | metabolism |
| 31 | 2.24 | gi\|19747198 | Sus scrofa clone RP44-326F1. | 1e−27 | | | unknown |
| 32 | 2.16 | gi\|2226003 | Human Tigger1 transposable element. | 3e−06 | human\| | 4.40E−08 | differen- |
| | | | | | BI057315 | | tiation |
| 33 | 2.01 | gi\|9910143 | H. sapiens beta | 0 | | | metabolism |
| | | | 1,3-galactosyltransferase (C1GALT1), | | | | |
| | | | mRNA | | | | |
| | | lower in 4 wk | | | | | |
| | | acc. number | | | | | |
| 1 (10) | −3.63 | emb\|Z69585.1\| | S. scrofa mRNA for glutathione | 0 | | | metabolism |
| | | | S-transferase | | | | |
| 2 (9) | −3.59 | emb\|Z69586.1\| | S. scrofa mRNA for glutathione | 0 | | | metabolism |
| | | | S-transferase | | | | |
| 3 | −3.78 | gb\|AC007281.3\| | Homo sapiens BAC clone | 9E−17 | | | unknown |
| | | | RP11-457F14 from 2. | | | | |
| 4 (2) | −3.01 | gb\|AC017079.5\| | Homo sapiens BAC clone | 5.00E−05 | human\| | 2.40E−15 | unknown |
| | | | RP11-462M9 from 2, complete | | THC1894090 | | |
| | | | sequence | | | | |
| 5 | −2.79 | gb\|AF027386.1\| | Bos taurus glutathione-S-transferase. | e−101 | | | metabolism |
| 6 (2) | −2.55 | gb\|L13068.1\| | Sus scrofa calbindin D-9k mRNA | 0 | | | metabolism |
| 7 | −2.97 | gi\|10432858\| | Homo sapiens cDNA FLJ11576 fis, | e−112 | | | unknown |
| | | | clone HEMBA1003548. | | | | |
| 8 | −3.10 | gi\|1185282\| | S. scrofa mRNA for glutathione | 0 | | | metabolism |
| | | | S-transferase | | | | |
| 9 (4) | −3.70 | gi\|163648\| | Bovine PTP (PAP) mRNA complete | e−162 | | | immune |
| | | | cds | | | | |
| 10 | −2.16 | gi\|17572809\| | Homo sapiens THO complex 4 | 1E−40 | | | metabolism |
| | | | (THOC4) | | | | |
| 11 | −2.12 | gi\|18767559\| | Homo sapiens BAC clone | 1E−23 | pig\|BF713657 | 8.10E−40 | unknown |
| | | | RP13-650L7 from 2, complete | | | | |
| | | | sequence | | | | |
| 12 | −2.12 | gi\|2581789\| | Mesocricetus auratus cytochrome c | 3E−22 | | | metabolism |
| | | | oxidase chain I and II | | | | |
| 13 | −3.06 | gi\|2887430\| | Homo sapiens KIAA0428 mRNA, | 0 | pig\|TC105467 | 0 | unknown |
| | | | partial cds | | | | |
| 14 (4) | −2.60 | gi\|37182060\| | Human clone DNA59613 | 2.00E−06 | pig\|TC153096 | 2.10E−87 | metabolism |
| | | | phospholipase inhibitor (UNQ511) | | | | |
| | | | mRNA | | | | |
| 15 | −2.56 | gi\|40254892\| | Homo sapiens hypothetical protein | 8E−13 | pig\|TC109417 | 3.00E−79 | unknown |
| | | | FLJ11273 (FLJ11273), mRNA | | | | |
| 16 | −2.20 | gi\|4758711\| | Homo sapiens maltase-glucoamylase | e−142 | | | metabolism |

TABLE 10
Differential expression if I-FABP and PAP as established by
microarray (m) and Northern blot (nb).

| segment pair | I-FABPm | I-FABPnb | PAPm | PAPnb |
|---|---|---|---|---|
| 5 | 1.2 | 1.0 | 0.3 | 2 |
| 6 | 2.1 | 1.5 | 45 | 50 |
| 7 | 0.6 | 1.8 | 32 | 60 |
| 8 | 0.7 | 1.0 | 180 | 40 |

### EXAMPLE 3

The early transcriptional response of pig small intestinal mucosa to infection by Salmonella enterica serovar Typhimurium DT 104 analyzed by cDNA microarray.

Introduction

Salmonella species are a leading cause of human bacterial gastroenteritis. Although there is extensive molecular knowledge on the pathogen itself, understanding of the molecular mechanisms of host-pathogen interaction is limited. There is increasing evidence about Salmonella interaction with isolated cells or cell lines (macrophages and enterocytes) on the molecular level, however, very little is known about the complex interaction with multiple cell types present in the intestinal mucosa in vivo.

In the present study, we focus on bacterial invasion as an important step in the early interaction of Salmonella with the small intestinal mucosa in a pig model. Small intestinal segments are perfused with or without S. enterica serovar Typhimurium DT104, and whole mucosal scrapings were taken at zero, two, four, and eight hours. Immune histologically, subepithelial Salmonella was demonstrated at two hours and after in all jejunal and ileal locations. Jejunal mucosal gene expression analysis by a pig cDNA small intestinal microarray showed a limited number of up-regulated genes at four and eight hours: a transient response of IL8 and TM4SF20 at four hours, a sustained elevated level of MMP-1 (at four hours and eight hours), and the anti-inflammatory PAP showing the most pronounced response (at four hours and eight hours). Two other genes reacted at eight hours only.

Comparison with in vitro results suggests IL8 to originate from both enterocytes and macrophages, and MMP-1 from macrophages. PAP is of enterocyte origin and not described before in Salmonella infections. The magnitude of the PAP response suggests its importance, possibly in the defense against gram-negative bacteria.

These are the first microarray data on Salmonella-host interaction with whole in vivo mucosa. Most striking is the limited reaction at the jejunal level when compared to enterotoxic E. coli infection. It is concluded that this is probably due to the fact that Salmonella is well adapted to evade strong host responses.

In the present study, we describe the early transcriptional response of pig intestinal mucosa to invasion with S. typhimurium in the small intestinal perfusion technique (Niewold et al., 2005) using a pig intestinal cDNA microarray.

Materials & Methods

Animals

Pigs used for the SISP technique were purchased from purchased from a commercial piggery and were cross-bred Yorkshire×(Large White×Landrace). The animal experiment was approved by the local Animal Ethics Commission in accordance with the Dutch Law on Animal Experimentation. Animals were checked for Salmonella-free status by culturing feces samples ten days previous to the start of the experiment.

Bacterial Strain

The Salmonella strain used was an isolate from a field case of enterocolitis and was typed as Salmonella enterica serovar Typhimurium DT104.

SISP Technique

The SISP was performed essentially according to Niewold et al., 2005. Briefly, four pigs (six to seven weeks old) were sedated with 0.1 ml azaperone (Stressnil) per kg bodyweight. After 15 minutes, inhalation anesthesia was initiated with a gas mixture of 39% oxygen, 58% nitrous oxide and an initial 3% isoflurane; after ten minutes, 2% isoflurane. The abdominal cavity was opened and four pairs of small intestinal segments were prepared by inserting a small inlet tube in the cranial site of a segment and by inserting a wide outlet tube into the caudal site of a segment. Seven intestinal segments were prepared. The first two segments were located in the proximal jejunum directly after the ligament of Treitz. Segments three and four were located in the mid jejunum, and segments five, six and seven cover most of the ileum. The odd numbered segments (initially 40 cm) were perfused for one hour with peptone solution containing 10⁹ CFU/ml of S. typhimurium, followed by perfusion with peptone only. Control segments (numbered 2, 4, 6) (initially 20 cm) were perfused with peptone only. Mucosal samples for histology and RNA isolation (10 cm) were taken at zero, two, four, and eight hours, the tubing reconnected, and perfusion resumed. Perfusion was performed manually with syringes attached to the cranial tubes, 2 ml every 15 minutes. After perfusion, the pigs were euthanized by barbiturate overdose. Mucosal scrapings were taken for genomic analysis from four animals.

Isolation of Total RNA

Approximately 1 gram of frozen tissue (mucosal scrapings) was collected from SISP segments at several time points (see above), frozen in liquid nitrogen, and stored at −70° C. Tissue was homogenized directly in 10 ml TRIzol® reagent (GibcoBRL). After homogenization, insoluble material was removed by centrifugation at 12,000×g for ten minutes at 4° C. Further extraction of RNA from these homogenates was performed according to instructions of the manufacturer of TRIzol® reagent. The crude RNA pellet obtained from this isolation procedure was dissolved in 1 ml RNase-free water and precipitated with 0.25 ml of isopropanol and 0.25 ml of 0.8 M sodium citrate/1.2 M NaCl to remove proteoglycan and polysaccharide contamination. After centrifugation at 12,000×g for ten minutes at room temperature, RNA pellets were washed with 75% (v/v) ethanol and dissolved in RNase-free water. Subsequently, the RNA was treated with DNase, extracted with phenol-chloroform, and precipitated with ethanol. RNA pellets were washed with 75% (v/v) ethanol, dissolved in RNase-free water, and stored at −70° C. until further use. The integrity of the RNA was checked by analyzing 0.5 μg on a 1% (w/v) agarose gel.

Microarray Analysis

The microarray used was constructed from pig jejunal cDNA as described earlier (Niewold et al., 2005). cDNA probes and dual-color labeling, and hybridizations of microarray slides were performed as described earlier (Niewold et al., 2005), using the RNA MICROMAX TSA labeling and detection kit (PerkinElmer). The TSA probe labeling and array hybridization were performed as described in the instruction manual with minor modifications. The cDNA synthesis time was increased to three hours at 42° C. Briefly, oligo-dT primed biotin- (BI-) or fluorescein- (FL-) labeled cDNA was generated in a reversed transcriptase (RT) reaction using 1 or 2 μg of total RNA as template. The microarray was pre-hybridized in 5% SSC, 0.1% SDS and 1% BSA at 42° C. for 30 minutes. Subsequently, a microarray slide was simultaneously hybridized with both the BI- and FL-labeled preparations. Post-hybridization washes were performed according to the manufacturer's recommendations. BI- and FL-labeled cDNAs hybridized to the spots were sequentially detected with the fluorescent reporter molecule Cy5 (red) and Cy3 (green), respectively. In a second hybridization experiment, the labels were reversed (dye swap). Scanning for Cy5 and Cy3 fluorescence was performed in a Packard Bioscience BioChip Technologies apparatus (PerkinElmer). Image analysis was performed using the Scanarray™-express software (PerkinElmer). Reports were used for subsequent data analyses.

Data Analysis

After background correction, the data presented in an M/A plot were M=log₂R/G and A=log₂√(R×G). An intensity-dependent normalization was performed using the lowest function in the statistical software package R. The normalization was done with a fraction of 0.2 on all data points. For each EST, eight values were obtained, four for one slide and four for the dye-swap. Genes with three or more missing values were removed from further analysis. Missing values were possible due to a bad (local) signal-to-noise ratio. A gene was considered to be differentially expressed when the mean value of the ratio was >2 or <−2 and the cDNA was identified with significance analysis of microarrays (based on SAM with a False discovery rate <2%). Significant expression corresponds to a more than four-fold up- or down-regulation, respectively.

Immune Histology

Invasion was established by immune histology on deparaffinized tissue sections, using a specific anti-O anti-Salmonella antibody.

Results

Immune histologically, S. typhimurium was found subepithelially in all three (jejunal and ileal) locations at two, four, and eight hours. Similar patterns were observed in proximal and mid jejunum and ileum. The SISP procedure itself led to increasing histological edema and cellular infiltration.

Mid jejunal mucosal gene expression analysis by a pig cDNA small intestinal microarray showed that comparing with time zero hour, no down-regulated genes were found, nor any up-regulated genes at two hours. Seven different genes were up-regulated at four and eight hours. Up-regulated transcripts could be grouped into different reaction patterns, at four hours only, at both four hours and eight hours, and at eight hours only. Interleukin 8 (a chemoattractant and activator of neutrophils) and a transcript homologous to Homo sapiens TM4SF20 (of unknown function) showed a transient response at four hours. An additional three genes showed differential expression at both four hours and eight hours, Matrix metalloproteinase-1 (MMP-1), Pancreatitis-associated protein (PAP), and Cytochrome P450 (CytP450). Two transcripts showed a response at eight hours only (THOC4 and STAT3), which are involved in transcriptional control. Comparison of differential expression in infected segments between eight hours and zero hours, showed that CytP450 was up-regulated by the SISP procedure itself (Table 1).

Elucidation of the mechanisms involved in invasion of pathogens into the host is important for the rational design of prevention and treatment of infection and disease.

There is evidence to indicate that the ileum is a major site of invasion of Salmonella but the more proximal sites have not been studied as yet (Darwin and Miller, 1999). In most animal models, researchers have looked histologically at ileum and colon, and the time points sampled are usually days rather than hours. Only the ligated loop technique in rabbit and in guinea pig histological data are available from earlier events (as summarized by Darwin and Miller, 1999). Furthermore, using the ligated loop technique in pigs, ultrastructural invasion of Salmonella was shown to occur within minutes (Meyerholz et al., 2002). Whereas there obviously is histological information on in vivo S. typhimurium invasion, data on the molecular cellular responses are limited to infection experiments using isolated cells or cell lines.

In the present study, we have chosen to use the pig model because of the importance of SeT in pigs and because it is a good model for humans. The Small Intestinal Segment Perfusion (SISP) technique was chosen because in this model, the intestines have intact blood flow, innervation, and (as opposed to the ligated loop) luminal flow. Furthermore, the system allows for sampling at various time points and at different parts of the small intestine. After analysis by immune histology, invasion in jejunum and ileum appeared to be quite similar. It was decided to use the material of mid jejunum for a first genomic analysis because this enabled us to compare with the reaction to infection with enterotoxic E. coli, a non-invasive close relative of Salmonella. Furthermore, since jejunum is cranial from the ileum, it would probably be more important in terms of first reaction.

In our model, S. typhimurium appeared to invade very quickly in all three (jejunal and ileal) locations. Similar patterns were observed in proximal and mid jejunum and ileum. Immune histologically, S. typhimurium was demonstrated in a subepithelial location within two hours. The SISP procedure itself led to increasing histological edema and cellular infiltration, which is probably caused by the repeated handling of the intestines required to obtain the samples on successive time points. In terms of gene expression though, the effect of the procedure itself remained limited to expression of CytP450. Apart from the latter, no histological alterations could be seen. The absence of other significant histological changes in cell type distribution was corroborated by the absence of differential expression of I-FABP, an epithelial marker that we use as a standard for epithelial content (Niewold 2005).

Mucosal gene expression analysis by a pig cDNA small intestinal microarray showed including (CytP450) that S. typhimurium infection induced seven different up-regulated genes at four and eight hours. No down-regulated genes were found. Up-regulated transcripts could be grouped into different reaction patterns, early transient (four hours only), four hours and eight hours either constant or increasing, and late, i.e., at eight hours only. Interleukin 8 (a chemoattractant and activator of neutrophils) showed a transient response at four hours only, as did a transcript homologous to Homo sapiens TM4SF20, of unknown function.

Apart from CytP450, a further two genes showed differential expression at four hours and eight hours. Matrix metalloproteinase-1 (MMP-1) had a similar elevated level at four hours and eight hours. Pancreatitis-associated protein (PAP) showed at four hours a response similar to that of MMP-1, but increased even further at eight hours. Comparison with in vitro results obtained with Salmonella spp. suggests IL8 to originate from enterocytes (Eckman et al., 2000; Hobbie et al., 1997) and macrophages (Nau et al., 2002), and MMP-1 from macrophages (Nau et al., 2002). MMP-1 was also found expressed by intestinal fibroblasts (Salmela et al., 2002) in inflammatory conditions. MMP-1 is important in tissue remodeling.

PAP is of enterocyte origin and probably involved in the control of bacterial proliferation. A similar reaction of PAP was seen in our previous experiments with ETEC in the SISP technique. The magnitude of the PAP response suggests an important role in the innate defense, possibly against (gram-negative) bacteria. Given the striking response, it is surprising that PAP was not described before in Salmonella infections in, for instance, cell lines. However, data are very limited thus far, and the absence of a PAP response in the HT29 cell line (Eckmann et al., 2000) could also be due to its absence from the array used, alternatively, HT29 could be defective.

Furthermore, two transcripts showed a response at eight hours only. These genes are involved in transcriptional control. Comparing with in vitro results obtained with enterocytes, only a limited number of genes are found up-regulated, whereas the magnitude of reaction is much greater in the SISP. Another difference is that in vitro in HT29 cells (Eckmann et al., 2000), both up- and down-regulated genes were found, whereas we found no down-regulated genes. In macrophages (Rosenberger et al., 2000), expression differences of a larger magnitude were found. Based on this, it is tempting to suggest that the larger magnitude responses in our system are attributable to the macrophage population, however, the largest response in the SISP is from PAP, which is of clear enterocyte origin.

Concerning the limited amount of genes found, one of the reasons could be that in vivo relevant gene expression could be diluted due to the presence of a multitude of cell types (Niewold et al., 2005), in contrast to the homogeneous cell line. Second, relevant genes could be absent from the microarray.

Whereas it is possible that in out system genes are absent or that lower magnitude reactions are missed due to dilution, the fact is that using the same array and E. coli, at least 100 relevant genes did react (Niewold et al., 2005), which is an indication for the validity of the array.

This shows that the difference in reaction is not due to the microarray itself, or in the amount of bacteria, but is due to a difference in the nature and magnitude of the stimulus between E. coli and Salmonella. In the case of Salmonella, only part of the number of bacteria participates in invasion (Darwin and Miller, 2002), which is consistent with a lower stimulus. Alternatively, or in addition, S. typhimurium is well adapted to not evoke strong host responses. This is also consistent with the fact that no down-regulated genes were found, in contrast with ETEC. In the latter, the strong up-regulation necessitates cells to redirect resources, resulting in compensatory down-regulation.

Differentially expressed genes during Salmonella invasion. Sequences of the inserts of library clones (ID) were compared with the NCBI non-redundant (nr) database using blast(n) and the porcine and human EST databases (TIGR) using WU-BLAST 2.0 (blast(n) option). The accession (acc.) number of the nucleotide sequence (mRNA or DNA) that scored the highest degree of homology (lowest E-value) is listed (gene name). The number of additional library clones that aligned to an identical accession number is given in parentheses behind the ID of the clones that scored the lowest E-value. Based on the annotation in the databanks, a (tentative) function is given.

TABLE 11

| Ratio | | | | | | | |
|---|---|---|---|---|---|---|---|
| infected/ | | | | | | | |
| control | | | control 8 h/0 h | | | | |
| 2 h | 4 h | 8 h | 8 h | Accession nr | Gene name | E-value | (tentative) function |
| | 9 | 10 | | gi: 13027798 | H. sapiens matrix metalloproteinase 1 | 2.00E−22 | tissue remodeling |
| | | | | | (interstitial collagenase) (MMP1) | | |
| | 8 | 41 | | gi: 189600 | H. sapiens pancreatitis associated protein | e−162 | innate defense |
| | | | | | (PAP) | | |
| | 5 | | | gi: 47523123 | S. scrofa Interleukin 8 | 0 | innate defense |
| | 4 | | | gi: 13376165 | H. sapiens transmembrane 4 L six family | 7.00E−23 | unknown |
| | | | | | member 20 (TM4SF20) | | |
| | | 4 | | gi: 55770863 | H. sapiens THO complex 4 (THOC4) | 1.00E−40 | transcription |
| | | 5 | | gi: 47080104 | H. sapiens signal transducer and activator of | e−169 | transcription |
| | | | | | transcription 3 (STAT3) | | |
| | 2 | 2 | 13 | gi: 47523899 | S. scrofa cytochrome P450 3A29 (CYP3A29) | 0 | metabolism |

### EXAMPLE 4

### The Early Transcriptional Response to Experimental Rotavirus Infection in Germfree Piglets

Seven germ-free piglets, obtained by caesarean section from sows with a Great Yorkshire and Large White background, were housed in germ-free isolators at the animal facilities of the Animal Sciences Group in Lelystad, NL. Animals were fed sterilized coffee milk until day 18, and from then on, with irradiated pig pellets. On day 21, three animals were sacrificed (control), and four others were infected orally with 2×10⁵ rotavirus (strain RV277) particles/animal. Two animals were sacrificed at 12 hours post-infection (p.i.), the two remaining at 18 hours p.i. Of all animals, jejunal mucosal scrapings were taken for microarray analysis. Samples of controls (3), 12 hours p.i. (2), and 18 hours p.i. (2) were pooled separately, and differential expression of infected versus control was determined using the pig intestinal microarray described earlier (Niewold et al., 2005).

A gene was considered to be differentially expressed when the mean value of M was >2 or <−2 and the cDNA was identified with significance analysis of microarrays with a q-value of <2%. This q-value or False discovery rate is familiar to the “p-value” of T-statistics. Because a ratio is expressed in a log₂ scale, a ratio of >2 or <−2 corresponds to a more than four-fold up- or down-regulation, respectively.

Genes differentially expressed at 12 and 18 hours post-infection (p.i.). Sequences of the inserts of library clones (ID) were compared with the NCBI non-redundant (nr) database using blast(n) and the porcine and human EST databases (TIGR) using WU-BLAST 2.0 (blast(n) option). The accession (acc.) number of the nucleotide sequence (mRNA or DNA) that scored the highest degree of homology (lowest E-value) is listed (gene name). The number of additional library clones that aligned to an identical accession number is given in parentheses behind the ID of the clones that scored the lowest E-value. T(H)C number; accession number of tentative consensus sequence of Expressed Sequence Tags posted in the TIGR human (THC) and pig (TC) databases. T(H)C numbers are given when their E-value is lower than the E-value scored by comparison with the NCBI nr database. M; ratio of differential expression (log₂ scale).

TABLE 12

| | | Blast(n)/nr or refseq_rna database | | | WU-BLAST 2.0/TIGR | |
|---|---|---|---|---|---|---|
| ID (n) | M | acc. number | Gene name | E-value | T(H)C number | E-value |

lower in
infected

12 hours p.i.

| | | | | | | |
|---|---|---|---|---|---|---|
| 1 | 4.70 | gi: 31343156 | Bos taurus thioredoxin mRNA. | 0 | | |
| 2 (3) | 3.39 | gi: 47523893 | Sus scrofa cytochrome P450 2C49 | 0 | | |
| | | | (CYP2C49), mRNA | | | |
| 3 (5) | 3.06 | gi: 47523899 | Sus scrofa cytochrome P450 3A29 | 0 | | |
| | | | (CYP3A29), mRNA | | | |
| 4 | 2.87 | gi: 31657133 | H. sapiens fyn-related kinase (FRK), | 0 | | |
| | | | mRNA | | | |
| 5 | 1.82 | gi: 34782973 | H. sapiens cytochrome b reductase 1, | 6.00E−28 | THC2397584 | 2.20E−61 |
| | | | mRNA | | | |
| 6 (4) | 1.85 | gi: 164674 | Pig Na+/glucose cotransporter protein | E−150 | | |
| | | | (SGLT1) mRNA, 3′ end | | | |
| 7 | 1.68 | gi: 12025666 | H. sapiens chloride channel, calcium | 4.00E−91 | pig\|TC157231 | 1.30E−104 |
| | | | activated, family member 4 | | | |
| 8 | 1.63 | gi: 20381190 | lactase-phlorizin hydrolase | 1.00E−57 | | |
| | | | (Lactase-glycosylceramidase) | | | |

18 hours p.i.

| | | | | | | |
|---|---|---|---|---|---|---|
| 1 | 3.49 | gi: 29602784 | Sus scrofa cytochrome b (cytb) gene. | 2.00E−68 | pig\|TC219497 | 4.10E−79 |
| 2 | 3.08 | gi: 47523893 | Sus scrofa cytochrome P450 2C49 | 0 | | |
| | | | (CYP2C49), mRNA | | | |
| 3 (2) | 2.89 | gi: 5835873 | Blast-X >>>NADH dehydrogenase | 3.00E−55 | | |
| | | | subunit 5 [Sus scrofa ] | | | |
| 4 | 2.62 | gi: 42794753 | H. sapiens acyl-CoA synthetase | 0 | | |
| | | | long-chain family member 3 mRNA | | | |
| 5 | 2.59 | gi: 47523149 | Sus scrofa tear lipocalin (LCN1), | 1.00E−20 | pig\|TC149619 | 4.40E−109 |
| | | | mRNA | | | |
| 6 (4) | 2.45 | gi: 52851461 | H. sapiens mRNA for HUMAN | 3.00E−44 | pig\|TC129860 | 3.80E−91 |
| | | | UDP-glucuronosyltransferase 2B17 | | | |
| 7 | 2.43 | gi: 32189367 | H. sapiens immunoglobulin J | 3.00E−41 | pig\|TC134330 | 2.00E−77 |
| | | | polypeptide mRNA | | | |
| 8 | 2.38 | gi: 23242900 | H. sapiens hypothetical protein | 7.00E−24 | pig\|TC159234 | 5.20E−119 |
| | | | FLJ22800, mRNA | | | |
| 9 | 2.28 | gi: 14916240 | H. sapiens BAC clone RP11-455G16 | 3.00E−10 | THC2262345 | 5.80E−20 |
| | | | from 4 | | | |
| 10 | 2.25 | gi: 14346089 | Human DNA sequence from clone | 7.00E−05 | human\|AI369860 | 3.40E−05 |
| | | | RP11-413P11 | | | |
| 11 (20) | 2.23 | gi: 10938019 | H. sapiens fatty acid binding protein 2, | ######## | | |
| | | | intestinal | | | |
| 12 | 2.20 | gi: 7688976 | H. sapiens DKFZp564J157 protein | 2.00E−49 | pig\|TC128460 | 3.10E−127 |
| 13 | 2.06 | gi: 34782973 | H. sapiens cytochrome b reductase 1, | 6.00E−28 | THC2397584 | 2.20E−61 |
| | | | mRNA | | | |
| 14 (3) | 2.02 | gi: 164674 | Pig Na+/glucose cotransporter protein | 0 | | |
| | | | (SGLT1) mRNA, 3′ end. | | | |
| 15 | 1.82 | gi: 56711297 | H. sapiens hypothetical protein | E−175 | pig\|TC115986 | 4.00E−110 |
| | | | LOC51057 (H.loGene: 12438) | | | |

higher in
infected

12 h p.i.

| | | | | | | |
|---|---|---|---|---|---|---|
| 1 | 3.23 | gi: 40254892 | H. sapiens hypothetical protein | 1.00E−15 | pig\|TC137797 | 2.50E−37 |
| | | | FLJ11273 (FLJ11273), mRNA | | | |
| 2 (3) | 3.16 | gi: 57097500 | Canis familiaris similar to | 4.00E−33 | pig\|TC153096 | 2.10E−87 |
| | | | phospholipase inhibitor (LOC482701), | | | |
| | | | mRNA | | | |
| 3 (2) | 2.80 | gi: 32396225 | Bos taurus mucus-type core 2 | 0 | | |
| | | | beta-1,6-N-acetylglucosaminyl- | | | |
| | | | transferase mRNA | | | |
| 4 | 2.72 | gi: 50470950 | Zebrafish DNA sequence from clone | 3.00E−06 | cattle\|TC272801 | 7.00E−44 |
| | | | DKEY-89P3. | | | |
| 5 | 2.67 | gi: 31873567 | H. sapiens mRNA; cDNA | 2.00E−08 | human\|THC1931910 | 2.90E−23 |
| | | | DKFZp686L21223 | | | |
| 6 (7) | 2.65 | gi: 4758711 | H. sapiens maltase-glucoamylase | 0 | | |
| | | | (alpha-glucosidase) (MGAM), mRNA | | | |
| 7 | 2.41 | gi: 51591908 | Rattus norvegicus type I keratin KA13 | 7.00E−06 | human\|THC1945423 | 4.60E−14 |
| | | | (Ka13), mRNA | | | |
| 8 (4) | 2.31 | gi: 27894336 | H. sapiens keratin 20 (KRT20), | 3.00E−59 | | |
| | | | mRNA | | | |
| 9 (3) | 2.23 | gi: 57977284 | Pan troglodytes actin, beta (ACTB), | 3.00E−38 | | |
| | | | mRNA. | | | |
| 10 (4) | 2.21 | gi: 163648 | Bovine pancreatic thread (associated) | e−162 | | |
| | | | protein (PTP or PAP) mRNA | | | |
| 11 | 2.19 | gi: 17572809 | H. sapiens THO complex 4, mRNA | 1.00E−40 | | |
| 12 | 2.15 | gi: 27526530 | H. sapiens mRNA diff. expressed in | 2.00E−04 | pig\|BF713657 | 2.00E−40 |
| | | | malign. melanoma, clone MM D3 | | | |
| 13 | 2.02 | gi: 47523773 | Sus scrofa spermidine/spermine | e−109 | | |
| | | | N-acetyltransferase (SAT), mRNA. | | | |
| 14 | 2.00 | gi: 4186144 | Sus scrofa mRNA for hypothetical | 0 | pig\|TC149845 | 6.90E−116 |
| | | | protein small intestine | | | |
| 15 | 1.60 | gi: 27526529 | H. sapiens mRNA diff. expressed in | 4.00E−05 | pig\|TC153096 | 1.50E−42 |
| | | | malign. melanoma, clone MM K2 | | | |
| 16 | 1.59 | gi: 13027798 | H. sapiens matrix metalloproteinase 1 | 2.00E−22 | human\|THC2315629 | 1.20E−27 |
| | | | (interstitial collagenase) (MMP1), | | | |
| | | | mRNA. | | | |

18 h p.i

| | | | | | | |
|---|---|---|---|---|---|---|
| 1 | 3.91 | gi: 40254892 | H. sapiens hypothetical protein | 1.00E−15 | pig\|TC137797 | 2.50E−37 |
| | | | FLJ11273 (FLJ11273), mRNA | | | |
| 2 | 3.83 | gi: 32396225 | Bos taurus mucus-type core 2 | 0 | | |
| | | | beta-1,6-N-acetylglucosaminyl- | | | |
| | | | transferase mRNA | | | |
| 3 (3) | 3.83 | gi: 57097500 | Canis familiaris similar to | 4.00E−33 | pig\|TC153096 | 2.10E−87 |
| | | | phospholipase inhibitor (LOC482701), | | | |
| | | | mRNA | | | |
| 4 | 3.19 | | No significant hits found | | pig\|TC146119 | 2.20E−149 |
| 5 | 2.90 | gi: 27526534 | H. sapiens mRNA diff. expressed in | 1.00E−06 | pig\|TC97603 | 5.10E−78 |
| | | | malign. melanoma, clone MM G4 | | | |
| 6 (4) | 2.91 | gi: 18490137 | H. sapiens guanylate binding protein | ######## | | |
| | | | 2, interferon-inducible, mRNA .. | | | |
| 7 (4) | 2.85 | gi: 47523773 | Sus scrofa spermidine/spermine | e−109 | | |
| | | | N-acetyltransferase (SAT), mRNA. | | | |
| 8 | 2.88 | | No significant hits found | | human\|THC2001683 | 1.40E−07 |
| 9 | 2.67 | gi: 23343684 | Sus scrofa clone RP44-363K13, | 5.00E−25 | pig\|CN159449 | 2.00E−40 |
| | | | complete sequence | | | |
| 10 | 2.61 | gi: 27526529 | H. sapiens mRNA diff. expressed in | 4.00E−05 | pig\|TC153096 | 1.50E−42 |
| | | | malign. melanoma, clone MM K2 | | | |
| 11 | 2.50 | gi: 31873567 | H. sapiens mRNA; cDNA | 2.00E−08 | human\|THC1931910 | 2.90E−23 |
| | | | DKFZp686L21223 | | | |
| 12 (2) | 2.49 | gi: 4186144 | Sus scrofa mRNA for hypothetical | 0 | pig\|TC149845 | 6.90E−116 |
| | | | protein small intestine | | | |
| 13 | 2.40 | gi: 10437783 | H. sapiens cDNA: FLJ21643 fis, clone | 2.00E−55 | pig\|TC133801 | 2.80E−104 |
| | | | COL08382 | | | |
| 14 | 2.38 | gi: 14790114 | H. sapiens caspase 3 (CASP3), | 0.003 | pig\|TC202066 | 7.10E−126 |
| | | | transcript variant beta, mRNA | | | |
| 15 | 2.38 | gi: 57085092 | Canis familiaris similar to seven | 7.00E−13 | pig\|TC201163 | 0.0043 |
| | | | transmembr. helix receptor | | | |
| | | | (LOC479238), mRNA. | | | |
| 16 | 2.15 | gi: 47523065 | Sus scrofa caspase-3 (CASP3), mRNA | 0 | | |
| 17 | 2.09 | gi: 23110943 | H. sapiens proteasome (prosome, | 0 | | |
| | | | macropain) subunit alpha type, 6 | | | |
| | | | mRNA | | | |
| 18 | 2.08 | gi: 17572809 | H. sapiens THO complex 4, mRNA | 1.00E−40 | | |
| 19 | 2.06 | gi: 51591908 | Rattus norvegicus type I keratin KA13 | 7.00E−06 | human\|THC1945423 | 4.60E−14 |
| | | | (Ka13), mRNA | | | |
| 20 | 2.01 | gi: 27894336 | H. sapiens keratin 20, mRNA | 2.00E−15 | | |
| 21 (4) | 1.89 | gi: 4758711 | H. sapiens maltase-glucoamylase | 0 | | |
| | | | (alpha-glucosidase) (MGAM), mRNA | | | |
| 22 | 1.79 | gi: 163648 | Bovine pancreatic thread (associated) | e−162 | | |
| | | | protein (PTP or PAP) mRNA | | | |
| 23 | 1.67 | gi: 17391364 | H. sapiens cell division cycle 42 (GTP | e−124 | | |
| | | | binding protein, 25 kDa), mRNA | | | |

### EXAMPLE 5

### Salmonella Susceptibility Affects Gene Expression in the Chicken Intestine

Poultry products are an important source for Salmonella enterica. An effective way to prevent food poisoning due to Salmonella would be to breed chickens resistant to Salmonella. Unfortunately, resistance to Salmonella is a complex trait with many factors involved.

To learn more about Salmonella resistance in young chickens, a cDNA microarray analysis was performed to compare gene expression levels between a Salmonella-susceptible and a more resistant chicken line. Newly hatched chickens were orally infected with Salmonella serovar Enteritidis. Since the intestine is the first barrier the bacteria encounters after oral inoculation, gene expression was investigated in the intestine, from day 1 until day 21 post-infection. Differences in gene expression between the susceptible and resistant chicken lines were found in control and Salmonella-infected conditions.

Gene expression differences indicated that genes that affected T-cells activation were regulated in the jejunum of susceptible chickens in response to the Salmonella infection, while the more resistant chicken line regulated genes that could be related with macrophage activation at day 1 post-infection.

At days 7 and 9 post-infection, most gene expression differences between the two chicken lines were identified under control conditions, indicating a difference in the intestinal development between the two chicken lines that might be linked to the difference in Salmonella susceptibility. The findings in this study have lead to the identification of novel genes and possible cellular pathways of the host involved in Salmonella resistance.

In this study, the gene expression profiles in the small intestines of a fast and a slow growing meat-type chicken line were compared in control and Salmonella-infected conditions. It was suggested that slow growing chickens are more resistant to Salmonella compared with fast growing ones (8). Indeed, we found differences in Salmonella susceptibility as well as differences in host gene expression between the lines. The gene expression differences found with the microarray were confirmed using quantitative reverse transcription (RT)-PCR.

Materials and Methods

Chickens

Two meat-type chicken lines, fast growing, S (susceptible) and slow growing, R (resistant), were used in the present study (Nutreco®, Boxmeer, NL). Eighty one-day-old chickens of each line (S and R) were randomly divided into two groups, 40 chickens each. After hatching, it was determined that birds were free of Salmonella.

Experimental Infection

Salmonella serovar Enteritidis phage type 4 (nalidixic acid resistant) was grown in buffered peptone water (BPW) overnight while shaking at 150 rpm. Of each chicken line, one group of one-day-old chickens was orally inoculated with 0.2 ml of the bacterial suspension containing 10⁵ CFU Salmonella serovar Enteritidis. The control groups were inoculated with 0.2 ml saline. Five chickens of each group were randomly chosen and sacrificed at days 1, 3, 5, 7, 9, 11, 15 and 21 post-infection.

Before euthanization, the body weight of each chicken was measured. Pieces of the jejunum were snap frozen in liquid nitrogen and stored at −70° C. until further analyses. The liver was removed and weighted and kept at 4° C. until bacteriological examination. The study was approved by the institutional Animal Experiment Commission in accordance with the Dutch regulations on animal experimentation.

Bacteriological Examination

For detection of Salmonella serovar Enteritidis, a cloacal swab was taken and, after overnight enrichment, it was spread on brilliant green agar+100 ppm naladixic acid for Salmonella determination (37° C., 18 to 24 hours). One gram of liver of each bird was homogenized in 9 ml BPM, serial diluted in BPW, and plated onto brilliant green agar with nalidixic acid for quantitative Salmonella serovar Enteritidis determination (37° C., 18 to 24 hours) by counting the colony-forming units.

Statistics

Variance analysis with two factors (time, line and their interaction) was performed on the log (CFU) measured in the liver. Calculations were performed in the statistical package Genstat 6. Also, a regression analysis over time points was done with the chicken line as experimental factor. The response variables were weight and log (CFU) on eight time points. The weights of the chickens were age-matched compared using the Student t test.

RNA Isolation

Pieces of the jejunum were crushed under liquid nitrogen. Fifty to 100 mg tissues of the different chicks were used to isolate total RNA using TRIzol reagent (Invitrogen, Breda, NL), according to instructions of the manufacturer with an additional step. The homogenized tissue samples were resuspended in 1 ml of TRIzol Reagent using a syringe and 21 gauge needle and passing the lysate through ten times. After homogenization, insoluble material was removed from the homogenate by centrifugation at 12,000×g for ten minutes at 4° C.

For the array, hybridization pools of RNA were made in which equal amounts of RNA from five different chickens of the same line, condition and time point were present.

Hybridizing of the Microarray

The microarrays were constructed as described earlier (34). The microarrays contained 3072 cDNAs spotted in triplicate from a subtracted intestinal library and 1152 cDNAs from a concanavalin A stimulated spleen library. All cDNAs were spotted in triplicate on each microarray. Before hybridization, the microarray was pre-hybridized in 5% SSC, 0.1% SDS and 1% BSA at 42° C. for 30 minutes. To label the RNA, the MICROMAX TSA labeling and detection kit (PerkinElmer, Wellesly, Mass.) was used. The TSA probe labeling and array hybridization were performed as described in the instruction manual with minor modifications. Biotin- and fluorescein-labeled cDNAs were generated from 5 μg of total RNA from the chicken jejunum pools per reaction.

The cDNA synthesis time was increased to three hours at 42° C., as suggested (11). Post-hybridization washes were performed according to the manufacturer's recommendations. Hybridizations were performed in duplicate with the fluorophores reversed. After signal amplification, the microarrays were dried and scanned for Cy5 and Cy3 fluorescence in a Packard Bioscience BioChip Technologies apparatus. The image was processed with Genepix pro 5.0 (Genomic Solutions, Ann Arbor, Mich.) and spots were located and integrated with the spotting file of the robot used for spotting. Reports were created of total spot information and spot intensity ratio for subsequent data analyses.

Analysis of the Microarray Data

A total of 64 microarrays were used in this experiment. For each of the eight time points, the following four comparisons were made using pools of RNA from five different chickens: line R control vs. line S control, line R Salmonella vs. line S Salmonella, line R control vs. line R Salmonella, and line S control vs. line S Salmonella. For each cDNA, six values were obtained, three for one slide and three for the dye swap. Genes with two or more missing values were removed from further analysis. Missing values were possibly due to a bad signal-to-noise ratio. A gene was considered to be differentially expressed when the mean value of the ratio log₂ (Cy5/Cy3) was >1.58 or <−1.58 and the cDNA was identified with significance analysis of microarrays (based on SAM (33)) with a False discovery rate <2%. Because the ratio was expressed in a log₂ scale, a ratio of >1.58 or <−1.58 corresponded to a more than three-fold up- or down-regulation, respectively. Bacterial clones containing an insert representing a differentially expressed gene were sequenced and analyzed using Seqman as described (35).

Results

Bacteriological Examination and Body Weight

In all the animals inoculated with Salmonella serovar Enteritidis, the Salmonella was detected in the caecal content. In contrast, Salmonella serovar Enteritidis was undetected in any of the control animals. The number of Salmonella serovar Enteritidis found in the liver of chickens from the susceptible (S) and resistant (R) line is presented in FIG. 1. In general, more Salmonella serovar Enteritidis is found in the S line (P=0.056). Regression analysis revealed that in the S line, the (log) CFU increased until day 7, after which the CFU decreased, while in the R line, the amount of CFU decreased from day 1. The (log) CFU are quadratically decreasing in time (P=0.02) for the S line and linearly decreasing (P=0.004) for the R line.

In the control situation, we did not detect differences in body weight between the S and the R line until day 9. From day 11 onwards, the chickens from the S line were heavier than the R line (P<0.05). In FIG. 2 is shown that the chickens from the S line had a higher weight gain depression after Salmonella infection compared to the chickens from the R line (P=0.007).

Gene Expression Differences Between the Chicken Lines

Changes in mRNA expression in the jejunum in response to infection with Salmonella were compared in both chicken lines at eight different time points. Genes used for further analysis needed to meet the following criteria: their expression was altered more than three-fold due to the Salmonella infection in only one of the two chicken lines and their expression differed more than three-fold between the chicken lines, either in the control situation or the Salmonella-infected situation. Most genes differing between the two chicken lines after the Salmonella infection were found at day 1. In the control situation, most differences between the chicken lines were found at day 9. After day 15, only a few differentially expressed genes were identified between the chicken lines in control and Salmonella-infected chickens.

Gene Expression Response at Day 1

In the susceptible chicken line, 13 up-regulated and two down-regulated genes were identified after the Salmonella infection, of which the expression was not regulated in the resistant chicken line (Table 1). These genes were equally expressed in both chicken lines under control conditions. Due to the gene regulation in the susceptible chicken line after infection, expression differences between the two chicken lines were found in the Salmonella-infected conditions.

In the resistant chicken line, three genes were up-regulated and six genes were down-regulated in response to Salmonella, while these genes were not regulated in the susceptible chicken line (Table 13). Two of these genes were up-regulated in the resistant chicken line after the Salmonella infection and, therefore, expression differences between the two chicken lines were found for these genes in the Salmonella-infected conditions. The remaining seven genes already differed in the control situation between the two lines. An interferon-induced protein was lower expressed in the resistant chicken line under the control situation. The TNF receptor, Rho GTPase-activating protein, similar to ORF2, similar to Carboxypeptidase M and two unknown genes were under the control conditions higher expressed in the resistant chicken line. In contrast to the control situation, in the Salmonella-infected situation, no expression differences between the two lines were found for these seven genes. This was due to the up- or down-regulation in response to Salmonella only in the resistant chicken line, while in the susceptible chicken line, no up- or down-regulation after the Salmonella infection was detected for these genes (Table 13).

Gene Expression at Days 7 and 9

Most differences in expression levels between the two chicken lines in the control situation were detected at day 9 post-infection. At this time point, 34 genes were identified with different expression levels under control conditions between the two lines. Furthermore, at day 9, these genes were regulated in response to Salmonella only in the resistant chicken line. Interestingly, 28 out of these 34 genes also differed at day 7 under control condition between the two chicken lines (Table 13). However, at day 7, no regulation of more than three-fold was found in either chicken line in response to the Salmonella infection.

Strikingly, the following nine genes differed in expression levels between the two chicken lines at days 7 and 9 in control conditions as well as at day 1 in Salmonella-infected conditions: similar to mannosyl (alpha-1,3-)-glycoprotein beta-1,4-N-acetylglucosarninyl-transferase, ikaros transcription factor, ZAP-70, CDH-1D and five uncharacterized genes. The expression differences between the chicken lines at day 1 were detected after the Salmonella infection instead of in the control situation as shown for days 7 and 9. At other time points, no expression differences of more than three-fold were found for these genes.

Confirmation of the Microarray Data

Validation of the microarray data was done with LightCycler RT-PCR because it is quantitative, rapid and requires only small amounts of RNA. The ikaros transcription factor and the gene similar to mannosyl (alpha-1,3-)-glycoprotein beta-1,4-N-acetylglucosarninyl-transferase (GnT-IV) were tested at days 1, 7 and 9. Unfortunately, at day 1, no expression differences could be found with the LightCycler for these genes, because the expression levels were below our detection limit. At days 7 and 9, the expression levels were higher in all groups and expression could be detected. With the LightCycler RT, (relative) concentrations of mRNA are measured, while the microarray detects expression differences. Therefore, the expression ratios between the two chicken lines were calculated for the control animals and the Salmonella-infected animals. For both tested genes, the results of the microarray were confirmed with the RT-PCR. The control animals of the resistant chicken line had higher expression levels for the two tested genes compared to the susceptible chicken line. After the Salmonella infection, no expression differences between the two chicken lines were found.

At day 1, distinct differences in gene expression were found comparing the two chicken lines. Differences in response to the Salmonella infection were found, as well as differences in the control situation of age-matched chickens.

In the susceptible chicken line, a number of uncharacterized genes was up-regulated in response to the Salmonella infection, as well as some known genes. One of these genes is the Ikaros transcription factor. Ikaros has an important function in T-cell development (14). ZAP-70 is another gene found at day 1 that is up-regulated in the susceptible chicken line. ZAP-70 plays a fundamental role in the initial step of the T-cell receptor signal transduction (6), and probably also plays an important role in growth and differentiation in several tissues including the intestine (10). CDH1-D, the third identified gene, has a role in the regulation of the cell cycle (37). Mannosyl (alpha-1,3-)-glycoprotein beta-1,4-N-acetylglucosaminyltransferase (GnT-IV) was also up-regulated at day 1 in the susceptible chicken line. GnT-IV is one of the key glycosyltransferases regulating the formation of highly branched complex type N-glycans on glycoproteins. GNT-IV is up-regulated during differentiation and development and highly expressed in leukocytes and T-cell-associated lymphoid tissues, like the small intestine (40). The inducible T-cell co-stimulator was the last known gene identified to be up-regulated at day 1 in response to Salmonella in the susceptible chicken line. The inducible co-stimulator is not expressed on naïve T-cells, but requires the activation of T-cells via the T-cell receptor (24). These findings suggest that T-cells are in another direction activated, maturated or more activated in the susceptible chicken line at day 1 due to the Salmonella infection compared to the resistant chicken line. It is in line with other findings, showing that an oral Salmonella enterica serovar Enteritidis infection increased the number of T-cells in the intestine, suggesting that a Salmonella infection either stimulated gut-associated T-cells to expand or recruited more T-cells to the mucosal tissues (29). Furthermore, expression of the CXC chemokines IL-8 and K60 was up-regulated in the jejunum of Salmonella serovar Typhimurium-infected chicken early after the infection (39). As CXC chemokines are chemoattractant for polymorphonuclear cells and naïve T-cells, this further confirms the role of T-cell activation in the early response to a Salmonella infection in Salmonella-susceptible chickens, while in the resistant chickens, other processes might be more dominant.

In contrast to the Salmonella-susceptible chickens, the resistant chickens did not up-regulate genes involved in T-cell activation in response to the infection. On the contrary, at day 1 post-infection, a TNF receptor was down-regulated in the resistant chicken line in response to Salmonella, while expression of this gene is strongly increased upon T-cell activation (21). In the control situation, this gene also differed in expression between the two chicken lines with higher expression in the resistant chicken line. CD4⁺ cells have a higher expression of this TNF receptor compared to CD8⁺ cells (21), so possibly the resistant chicken line has more CD4⁺ cells in the jejunum.

However, the chicken lines might also differ in the amount of macrophages, as expression of the TNF receptor is also shown in macrophages (30). This latter suggestion is supported by carboxypeptidase M, a macrophage differentiation marker (23), which is also higher expressed in the resistant chicken line in the control situation compared to the Salmonella-susceptible chicken line. After the Salmonella infection, carboxypeptidase M is down-regulated in the resistant chicken line as is the TNF receptor, so possibly the resistant chicken line has a different macrophage activation compared to the susceptible chicken line at day 1 post-infection.

Cytochrome P450 and apolipoprotein B were down-regulated at day 1 in the S line but not in the R line. They were also down-regulated in the susceptible chicken line when susceptibility to malabsorption syndrome was studied (35), a model for intestinal disturbances in young chickens. Down-regulation of apolipoprotein B and cytochrome P450 in intestinal epithelium was also shown in response to pro-inflammatory cytokines (2, 36). So the down-regulation of apolipoprotein B and cytochrome P450 might be a response to disturbances in the intestine, which in the susceptible line is thought to be more extensive.

At days 7 and 9 post-infection, gene expression differences in the control situation were detected between the S line and the R-line. As the S line grows faster than the R line, it is not surprising to find differences in the control situation at the intestinal level. From day 11 onwards, the weights of the healthy chickens from both lines differ significantly. The differences in gene expression at days 7 and 9 in the control situation reflect a difference in the development of the intestine of the young chickens. It is known that the morphology of the small intestine changes rapidly after hatch (7), but the early changes in intestinal morphology were not studied for chickens differing in growth rate. However, it is known that genetic selection on growth rate has effects on the intestinal structure of chickens of four weeks old (31).

Nine of the genes found at days 7 and 9 in the control situation also showed expression differences at day 1 after Salmonella infection. Five of these genes are uncharacterized, but the remaining four have a function in T-cell activation. The expression differences in the control situation at days 7 and 9 for these genes may be linked to the difference in stimulation of the immune system in the control situation of both chicken lines by microbes developing the gut flora in the young animals (9).

This study has revealed differences in gene expression in Salmonella-susceptible and -resistant chicken lines. Gene expression indicated that T-cells are more activated in the susceptible chicken line in response to the Salmonella infection, while the resistant chicken line had a better macrophage activation at day 1 post-infection.

Marked expression differences were also found for multiple uncharacterized genes. Although the precise function for most of the identified genes is yet unclear, these findings give possibilities to take disease susceptibility into account in breeding programs.

TABLE 13
Genes at day 1 with more than three-fold expression differences due to the Salmonella infection in only
one of the two chicken lines (S or R) and expression differences between the chicken lines either in the
control situation, or the Salmonella-infected situation.

| | | | | | | |
|---|---|---|---|---|---|---|
| Accession no. | Gene name | locus ID | S_{contr} − Sₛₐₗ^{a} | R_{contr} − Rₛₐₗ^{a} | S_{contr} − R_{contr}^{b} | Sₛₐₗ − Rₛₐₗ^{b} |

Regulated after Salmonella infection in susceptible chicken line

| | | | | | | |
|---|---|---|---|---|---|---|
| NM_001012824.1 | similar to mannosyl (alpha-1,3-)-glycoprotein | | +2.11 | 0 | 0 | +3.23 |
| | beta-1,4-N-acetylglucosaminyltransferase, | | | | | |
| | isoenzyme A; UDP-N-acetylglucosamine:alpha1 | | | | | |
| | (GnT-IV) | | | | | |
| Y11833.1 | GGIKTRF G. gallus mRNA for Ikaros transcription | | +2.01 | 0 | 0 | +3.61 |
| | factor | | | | | |
| XM_418206.1 | similar to Tyrosine-protein kinase ZAP-70 (70 kDa | 420086 | +1.61 | 0 | 0 | +2.99 |
| | zeta-associated protein) (Syk-related tyrosine | | | | | |
| | kinase) | | | | | |
| AJ719433.1 | mRNA for hypothetical protein, clone 2e14 | | +1.66 | 0 | 0 | +3.69 |
| CR387311.1 | finished cDNA, clone ChEST351c21 | | +1.78 | 0 | 0 | +3.38 |
| DN828706 | expressed sequence tag | | +1.74 | 0 | 0 | +3.69 |
| DN828699 | expressed sequence tag | | +1.97 | 0 | 0 | +2.82 |
| BU227174 | expressed sequence tag | | +1.92 | 0 | 0 | +2.86 |
| DN828707 | expressed sequence tag | | +2.59 | 0 | 0 | +3.47 |
| DN828697 | expressed sequence tag | | +1.62 | 0 | 0 | +2.89 |
| AF421549 | CDH1-D | | +2.22 | 0 | 0 | +3.58 |
| CR389073.1 | finished cDNA, clone ChEST347g18 | | +1.65 | 0 | 0 | +2.56 |
| XM_421959.1 | PREDICTED: similar to inducible T-cell | 424105 | +1.63 | 0 | 0 | +2.84 |
| | co-stimulator | | | | | |
| M18421 | apoB mRNA encoding apolipoprotein | 211153 | −1.62 | NA | 0 | −1.74 |
| NM_001001751.1 | cytochrome P450 A 37 (CYP3A37) | | −1.62 | 0 | 0 | −1.69 |

Regulated after Salmonella infection in resistant chicken line

| | | | | | | |
|---|---|---|---|---|---|---|
| CD726841.1 | expressed sequence tag | | 0 | +1.63 | 0 | −2.14 |
| XM_422715 | PREDICTED: similar to Fc fragment of IgG | 424904 | 0 | +1.58 | 0 | −2.64 |
| | binding protein; IgG Fc binding protein | | | | | |
| XM_421662.1 | PREDICTED: similar to Interferon-induced protein | 423790 | 0 | +2.03 | +1.63 | NA |
| | with tetratricopeptide repeats 5 (IFIT-5) (Retinoic | | | | | |
| | acid- and interferon-inducible 58 kDa protein) | | | | | |
| XM_417585.1 | PREDICTED: similar to tumor necrosis factor | 419424 | 0 | −1.66 | −1.81 | 0 |
| | receptor superfamily, member 18 isoform 3 | | | | | |
| | precursor; glucocorticoid-induced TNFR-related | | | | | |
| | protein; activation-inducible TNFR family | | | | | |
| | receptor; TNF receptor superfamily | | | | | |
| | activation-inducible protein | | | | | |
| XM_423002.1 | PREDICTED: similar to Rho GTPase-activating | 425219 | 0 | −1.68 | −1.82 | 0 |
| | protein; brain-specific Rho GTP-ase-activating | | | | | |
| | protein; rac GTPase activating protein; | | | | | |
| | GAB-associated CDC42; RhoGAP involved in the- | | | | | |
| | catenin-N-cadherin and NMDA receptor signaling | | | | | |
| DN828701 | expressed sequence tag | | 0 | −1.78 | −1.96 | 0 |
| BU457068.1 | cDNA clone ChEST200c16 | | 0 | −1.73 | −1.99 | 0 |
| XM_425603.1 | PREDICTED: Gallus gallus similar to ORF2 | 428036 | 0 | −1.9 | −2.08 | 0 |
| XM_416085.1 | PREDICTED: similar to Carboxypeptidase M | 417843 | 0 | −2.02 | −2.34 | 0 |
| | precursor | | | | | |

TABLE 14
Genes with more than three-fold expression differences due to the Salmonella infection in only one of
the two chicken lines (S or R) at day 1 and different expression levels between the two chicken lines
in the control situation at days 7 and 9.

| | | | day 7^{a} | | day 9^{a} | |
|---|---|---|---|---|---|---|
| accession no. | gene name | locus ID | contr. | inf. | contr. | inf. |
| NM_001012824.1 | similar to mannosyl (alpha-1,3-)-glycoprotein | | 1.95 | 0.71 | 2.75 | −1.05 |
| | beta-1,4-N-acetylglucosaminyltransferase, isoenzyme A; | | | | | |
| | UDP-N-acetylglucosamine:alpha1 (GnT-IV) | | | | | |
| Y11833.1\| | GGIKTRF G. gallus mRNA for Ikaros transcription factor | | 2.06 | 0.21 | 2.50 | −0.75 |
| XM_418206.1 | similar to Tyrosine-protein kinase ZAP-70 (70 kDa | 420086 | 2.33 | 0.26 | 2.73 | −0.80 |
| | zeta-associated protein) (Syk-related tyrosine kinase) | | | | | |
| AF421549 | CDH1-D | | 2.23 | 0.16 | 2.60 | −0.70 |
| AJ719433.1 | mRNA for hypothetical protein, clone 2e14 | | 2.23 | 0.37 | 2.73 | −0.87 |
| CR387311.1 | finished cDNA, clone ChEST351c21 | | 2.33 | 0.40 | 2.12 | −0.74 |
| DN828706 | expressed sequence tag | | 2.59 | 0.29 | 2.35 | −0.83 |
| DN828699 | expressed sequence tag | | 2.07 | 0.29 | 2.29 | −0.74 |
| BU227174 | expressed sequence tag | | 2.25 | 0.43 | 2.45 | −0.37 |
| XM_417797.1\| | PREDICTED: similar to protein tyrosine phosphatase 4a2 | 419649 | 1.78 | 0.39 | 2.03 | −0.91 |
| NM_001012914.1 | signal transducer and activator of transcription 4 (STAT4) | | 2.00 | 0.25 | 2.28 | −0.87 |
| XM_419701.1\| | PREDICTED: similar to T-cell activation Rho | 421662 | 2.30 | 0.33 | 2.19 | −0.79 |
| | GTPase-activating protein isoform b | | | | | |
| NM_001006289.1 | similar to 14-3-3 protein beta/alpha (Protein kinase C inhibitor | 419190 | 2.27 | 0.35 | 1.75 | −0.63 |
| | protein-1) (KCIP-1) (Protein 1054) | | | | | |
| NM_204417.1\| | protein tyrosine phosphatase, receptor type, C (PTPRC) | | 2.27 | 0.33 | 2.23 | −0.78 |
| XM_420925 | PREDICTED: similar to interferon-induced membrane protein | 422993 | 3.10 | −0.12 | 1.67 | 0.82 |
| | Leu-13/9-27 | | | | | |
| AJ725129 | riken1 cDNA clone 29g19s4, mRNA sequence | | 2.13 | 1.70 | 2.51 | −0.48 |
| AJ719476.1 | mRNA for hypothetical protein, clone 2k22 | | 2.18 | 0.50 | 1.76 | −0.65 |
| AJ719498.1 | mRNA for hypothetical protein, clone 2n23 | | 2.48 | 0.39 | 2.27 | −0.69 |
| AJ443170 | dkfz426 cDNA clone 33p14r1, mRNA sequence | | 2.55 | 0.44 | 1.92 | −0.82 |
| BU216613 | expressed sequence tag | | 2.46 | 0.57 | 1.84 | −0.66 |
| BU128188 | expressed sequence tag | | 3.17 | −0.20 | 1.82 | 0.64 |
| DN828698 | expressed sequence tag | | 2.07 | 0.48 | 1.73 | −0.47 |
| DN828705 | expressed sequence tag | | 1.64 | 0.24 | 2.44 | −0.48 |
| DN828700 | expressed sequence tag | | 2.12 | 0.37 | 1.77 | −0.48 |
| DN828703 | expressed sequence tag | | 2.13 | 0.35 | 2.21 | −0.88 |
| DN828702 | expressed sequence tag | | 2.18 | 0.42 | 1.67 | −0.76 |
| DN828704 | expressed sequence tag | | 2.25 | 0.29 | 1.74 | −0.51 |
| DN828696 | expressed sequence tag | | 2.52 | 0.36 | 2.07 | −0.84 |

TABLE 15
Ratio of the expression levels (resistant chickens/susceptible
chickens) found with the LightCycler RT-PCR and the microarray for
the ikaros transcription factor and the gene similar to mannosyl
(alpha-1,3-)-glycoprotein beta-1,4-N-acetylglucosaminyltransferase
(GnT-IV).

| | GnT-IV | | ikaros | |
|---|---|---|---|---|
| | microarray | RT-PCR | microarray | RT-PCR |
| Day 7 control | 3.9 | 4.2 | 4.2 | 2.4 |
| Day 9 control | 6.7 | 2.6 | 5.7 | 2.2 |
| Day 7 salmonella | 1.6 | 0.6 | 1.2 | 0.7 |
| Day 9 salmonella | 0.5 | 0.9 | 0.6 | 1.0 |

### REFERENCES

Altschul S. F., T. L. Madden, A. A. Schaffer, J. Zhang, Z. Zhang, W. Miller, and D. J. Lipman 1997. Gapped BLAST and PSI-BLAST: a new generation of protein database search programs. Nucleic Acids Res. 25:3389-3402.
Chuaqui R. F., R. F. Bonner, C. J. Best, J. W. Gillespie, M. J. Flaig, S. M. Hewitt, J. L. Phillips, D. B. Krizman, M. A. Tangrea, M. Ahram, W. M. Linehan, V. Knezevic, and M. R. Emmert-Buck 2002. Post-analysis follow-up and validation of microarray experiments. Nat. Genet. 32 Suppl:509-14.
Darwin K. H. and V. L. Miller 1999. Molecular basis of the interaction of Salmonella with the intestinal mucosa. Clin. Microbiol. Rev. 12:405-428.
Desjeux A., M. Barthet, S. Barthellemy, J. C. Dagom, P. Hastier, D. Heresbach, J. P. Bernard, and J. C. Grimaud 2002. Serum measurements of pancreatitis-associated protein in active Crohn's disease with ileal location. Gastroenterol. Clin. Biol. 26:23-28.
Diekgraefe B. K., W. F. Stenson, J. R. Korzenik, P. E. Swanson, and C. A. Harrington 2000. Analysis of mucosal gene expression in inflammatory bowel disease by parallel oligonucleotide arrays. Physio. Genomics 4:1-11.
van Dijk A. J., T. A. Niewold, M. J. A. Nabuurs, J. van Hees, P. De Bot, N. Stockhofe-Zurwieden, M. Ubbink-Blanksma, and A. C. Beynen 2002. Small intestinal morphology and disaccharidase activities in early-weaned piglets fed a diet containing spray-dried porcine plasma. J. Vet. Med. A 49:81-86.
Dudoit S., Y. H. Yang, M. J. Callow, and T. P. Speed 2002. Statistical methods for identifying differentially expressed genes in replicated cDNA microarray experiments. Statistica Sinica 12:111-139.
Eckmann L., J. R. Smith, M. P. Housley, M. B. Dwinell, and M. F. Kagnoff 2000. Analysis by high density cDNA arrays of altered gene expression in human intestinal epithelial cells in response to infection with the invasive enteric bacteria Salmonella. J. Biol. Chem. 275:14084-94.
Fahrenkrug S. C., T. P. Smith, B. A. Freking, J. Cho, J. White, J. Vallet, T. Wise, G. Rohrer, G. Pertea, R. Sultana, J. Quackenbush, and J. W. Keele 2002. Porcine gene discovery by normalized cDNA-library sequencing and EST cluster assembly. Mamm. Genome 8:475-478.
Glatz J. F., and G. J. van der Vusse 1996. Cellular fatty acid-binding proteins: their function and physiological significance. Progress in Lipid Res. 35:243-282.
Georges M. 2001. Recent progress in livestock genomics and potential impact on breeding programs. Theriogenology 55:15-21.
van Hemert S., B. H. Ebbelaar, M. A. Smits, and J. M. Rebel 2003. Generation of EST and microarray resources for functional genomic studies on chicken intestinal health. Anim. Biotechnol. 14:133-143.
Hobbie S., L. M. Chen, R. J. Davis, and J. E. Galan 1997. Involvement of mitogen-activated protein kinase pathways in the nuclear responses and cytokine production induced by Salmonella typhimurium in cultured intestinal epithelial cells. J. Immunol. 159:5550-5559.
Hooper L. V. and J. I. Gordon 2001. Commensal host-bacterial relationships in the gut. Science 292:1115-1118.
Hsiao L. L., R. V. Jensen, T. Yoshida, K. E. Clark, J. E. Blumenstock, and S. R. Gullans 2002. Correcting for signal saturation errors in the analysis of microarray data. Biotechniques 32:330-2, 334, 336.
Karsten S. L., V. M. D. Van Deerlin, C. Sabatti, L. H. Gill, and D. H. Geschwind 2002. An evaluation of tyramide signal amplification and archived fixed and frozen tissue in microarray gene expression analysis. Nucleic Acids Res. 30:E4.
Kelly D. and S. Conway 2001. Genomics at work: the global response to enteric bacteria. Gut 49:612-613.
Kiers J. L., M. J. R. Nout, F. M. Rombouts, M. J. A. Nabuurs, and J. van der Meulen 2001. Protective effect of processed soybean during perfusion of ETEC-infected small intestinal segments of early-weaned piglets. In: J. E. Linberg and B. Ogle (Eds), Digestive physiology of pigs. CABI Publishing, New York, pp. 261-263.
Lamont S. J. 1998. Impact of genetics on disease resistance. Poult. Sci. 77:1111-8.
Liu F. T., R. J. Patterson and J. L. Wang 2002. Intracellular functions of galectins. Biochim. Biophys. Acta. 1572:263-73.
Liu H. C., H. H. Cheng, V. Tirunagaru, L. Sofer and J. Burnside 2001. A strategy to identify positional candidate genes conferring Marek's disease resistance by integrating DNA microarrays and genetic mapping. Anim. Genet. 32:351-359.
Meltzer P. S. 2001. Spotting the target: microarrays for disease gene discovery. Curr. Opin. Genet. Dev. 11:258-263.
Meyerholz D. K., T. J. Stabel, M. R. Ackermann, S. A. Carlson, B. D. Jones, and J. Pohlenz 2002. Early epithelial invasion by Salmonella enterica serovar Typhimurium DT104 in the swine ileum. Vet. Pathol. 39:712-720.
Min W., H. S. Lillehoj, S. Kim, J. J. Zhu, H. Beard, N. Alkharouf and B. F. Matthews 2003. Profiling local gene expression changes associated with Eimeria maxima and Eimeria acervulina using cDNA microarray. Appl. Microbiol. Biotechnol. 62:392-399.
Montrose M. H. 2003. The future of GI and liver research: editorial perspectives: I. Visions of epithelial research. Am. J. Physiol. Gastrointest. Liver Physiol. 284:G547-550.
Moody D. E. 2001. Genomics techniques: an overview of methods for the study of gene expression. J. Anim. Sci. 79:E128-E135.
Morgan R. W., L. Sofer, A. S. Anderson, E. L. Bemberg, J. Cui, and J. Burnside 2001. Induction of host gene expression following infection of chicken embryo fibroblasts with oncogenic Marek's disease virus. J. Virol. 75:533-539.
Nabuurs M. J. A., A. Hoogendoorn, F. G. van Zijderveld, and J. D. van der Klis 1993a. A long-term perfusion test to measure net absorption in the small intestine of weaned pigs. Res. Vet. Sci. 55:108-114.
Nabuurs M. J. A., A. Hoogendoorn, E. van der Molen, and A. L. M. van Osta 1993b. Villus height and crypt depth in weaned and unweaned pigs, reared under various circumstances in NL. Res. Vet. Sci. 55: 78-84.
Nau G. J., J. F. Richmond, A. Schlesinger, E. G. Jennings, E. S. Lander, and R. A. Young 2002. Human macrophage activation programs induced by bacterial pathogens. Proc. Natl. Acad. Sci. U.S.A. 99:1503-8.
Neiman P. E., A. Ruddell, C. Jasoni, G. Loring, S. J. Thomas, K. A. Brandvold, R. Lee, J. Burnside and J. Delrow 2001. Analysis of gene expression during myc oncogene-induced lymphomagenesis in the bursa of Fabricius. Proc. Natl. Acad. Sci. U.S.A. 98:6378-6383.
Niewold T. A., H. H. D. Kerstens, J. van der Meulen, M. A. Smits, and M. M. Hulst 2005. Development of a porcine small intestinal cDNA microarray: characterization, and functional analysis of the response to enterotoxic E. coli. Vet. Immunol. Immunopathol. 105: 317-329.
Niewold T. A., M. Meinen, and J. van der Meulen 2004. Plasma intestinal fatty acid binding protein (1-FABP) concentrations increase following intestinal ischemia in pigs. Res. Vet. Sci. 77:89-91.
van Ommen B. and R. Stierum 2002. Nutrigenomics: exploiting systems biology in the nutrition and health arena. Curr. Opin. Biotechnol. 13:517-521.
Pelsers M. M., Z. Namiot, W. Kisielewski, A. Namiot, M. Januszkiewicz, W. T. Hermens, and J. F. Glatz 2003. Intestinal-type and liver-type fatty acid-binding protein in the intestine. Tissue distribution and clinical utility. Clin. Biochem. 36:529-535.
Rajeevan M. S., S. D. Vernon, N. Taysavang and E. R. Unger 2001. Validation of array-based gene expression profiles by real-time (kinetic) RT-PCR. J. Mol. Diagn. 3:26-31.
Reid G., M. E. Sanders, H. R. Gaskins, G. R. Gibson, A. Mercenier, R. Rastall, M. Roberfroid, I. Rowland, C. Cherbut, and T. R. Klaenhammer 2003. New scientific paradigms for probiotics and prebiotics. J. Clin. Gastroenterol. 7:105-118.
Roberts M. A., D. M. Mutch, and J. B. German 2001. Genomics: food and nutrition. Curr. Opin. Biotechnol. 12:516-522.
Rosenberger C. M., M. G. Scott, M. R. Gold, R. E. W. Hancock, and B. B. Finlay 2000. Salmonella typhimurium infection and lipopolysaccharide stimulation induce similar changes in macrophage gene expression. J. Immunol. 164:5894-5904.
Rothschild M. F. 2003. Advances in pig genomics and functional gene discovery. Comp. Funct. Genom. 4:266-270.
Ruchaud S., N. Korfali, P. Villa, T. J. Kottke, C. Dingwall, S. H. Kaufmann and W. C. Earnshaw 2002. Caspase-6 gene disruption reveals a requirement for lamin A cleavage in apoptotic chromatin condensation. EMBO J. 21:1967-77.
Salmela M. T., T. T. MacDonald, D. Black, B. Irvine, T. Zhuma, U. Saarialho-Kere, S. L. Pender 2002. Up-regulation of matrix metalloproteinases in a model of T cell mediated tissue injury in the gut: analysis by gene array and in situ hybridization. Gut 51:540-547.
Sambrook J., E. F. Fritsch, and T. Maniatis 1989. Molecular cloning: a laboratory manual. Cold Spring Harbor Laboratory, Cold Spring Harbor Press, NY (second edition).
Sellwood R., R. A. Gibbons, G. W. Jones and J. M. Rutter 1975. Adhesion of enteropathogenic Escherichia coli to pig intestinal brush borders: The existence of two pig phenotypes. J. Med. Microbiol. 8:405-411.
Smith E., L. Shi and G. Smith 2002. Expressed sequence tags for the chicken genome from a normalized ten-day-old white leghorn whole-embryo cDNA library. 3. DNA sequence analysis of genetic variation in commercial chicken populations. Genome 45:261-267.
Songserm T., B. Engel, D. J. van Roozelaar, G. L. Kok, A. Pijpers, J. M. A. Pol and A. A. H. M. ter Huurne 2002. Cellular immune response in the small intestine of two broiler chicken lines orally inoculated with malabsorption syndrome homogenates. Vet. Immunol. Immunopathol. 85:51-62.
Songserm T., J. M. Pol, D. van Roozelaar, G. L. Kok, F. Wagenaar and A. A. ter Huurne 2000. A comparative study of the pathogenesis of malabsorption syndrome in broilers. Avian Dis. 44:556-567.
Songserm T., B. Zekarias, D. J. van Roozelaar, R. S. Kok, J. M. A. Pol, A. Pijpers and A. ter Huurne 2002. Experimental reproduction of malabsorption syndrome with different combinations of reovirus, Escherichia coli, and treated homogenates obtained from broilers. Avian Dis. 46:87-94.
Stagliano K. E., E. Carchman and S. Deb 2003. Real-Time Polymerase Chain Reaction Quantitation of Relative Expression of Genes Modulated by p53 Using SYBR(R) Green I. Methods Mol. Biol. 234:73-92.
Tusher V. G., R. Tibshirani, and G. Chu 2001. Significance analysis of microarrays applied to the ionizing radiation response. Proc. Natl. Acad. Sci. U.S.A. 98:5116-21.
van der Heide L., D. Lutticken and M. Horzinek 1981. Isolation of avian reovirus as a possible etiologic agent of osteoporosis (“brittle bone disease”; “femoral head necrosis”) in broiler chickens. Avian Dis. 25:847-56.
van Hemert S., B. H. Ebbelaar, M. A. Smits and J. M. J. Rebel 2003. Generation of EST and microarray resources for functional genomic studies on chicken intestinal health. Animal Biotechnol. 14:133-143.
Verburg M., I. B. Renes, D. J. Van Nispen, S. Ferdinandusse, M. Jorritsma, H. A. Buller, A. W. Einerhand and J. Dekker 2002. Specific responses in rat small intestinal epithelial mRNA expression and protein levels during chemotherapeutic damage and regeneration. J. Histochem. Cytochem. 50:1525-1536.
Wallace J. L. 2001. Prostaglandin biology in inflammatory bowel disease. Gastroenterol. Clin. North. Am. 30:971-80.
Wintero A. K., M. Fredholm, and W. Davies 1996. Evaluation and characterization of a porcine small intestine cDNA library: analysis of 839 clones. Mamm. Genome. 7:509-517.
Xu J. and J. I. Gordon 2003. Honor thy symbionts. Proc. Natl. Acad. Sci. U.S.A. 100:10452-10459.
Yang Y. H., S. Dudoit, P. Luu, D. M. Lin, J. Peng, J. Ngai, and T. P. Speed 2002. Normalization for cDNA microarray data: a robust composite method addressing single and multiple slide systematic variation. Nucleic Acids Res. 30:e15.
Yonash N., L. D. Bacon, R. L. Witter and H. H. Cheng 1999. High resolution mapping and identification of new quantitative trait loci (QTL) affecting susceptibility to Marek's disease. Anim. Genet. 30:126-135.
You M., P. T. Ku, R. Hrdlickova and H. R. Bose, Jr. 1997. ch-IAP1, a member of the inhibitor-of-apoptosis protein family, is a mediator of the antiapoptotic activity of the v-Rel oncoprotein. Mol. Cel. Biol. 17:7328-41.
Yu J., M. I. Othman, R. Farjo, S. Zareparsi, S. P. MacNee, S. Yoshida and A. Swaroop 2002. Evaluation and optimization of procedures for target labeling and hybridization of cDNA microarrays. Mol. Vis. 8:130-7.
Zekarias B., T. Songserm, J. Post, G. L. Kok and J. M. Pol 2002. Development of organs and intestinal mucosa leukocytes in four broiler lines that differ in susceptibility to malabsorption syndrome. Poult. Sci. 81:1283-1288.
Zekarias B., A. Ter Huume, W. J. M. Landman, J. M. J. Rebel, J. M. A. Pol and E. Gruys 2002. Immunological basis of differences in disease resistance in the chicken. Vet. Res. 33:109-125.
van Zijderveld F. G., A. M. van Zijderveld-van Bemmel, and D. Bakker 1998. The F41 adhesin of enterotoxigenic Escherichia coli: inhibition of adhesion by monoclonal antibodies. Vet. Quart. 20:S73-78.
2. Bertilsson P. M., P. Olsson and K. E. Magnusson 2001. Cytokines influence mRNA expression of cytochrome P450 3A4 and MDRI in intestinal cells. J. Pharm. Sci. 90:638-46.
6. Chu D. H., C. T. Morita and A. Weiss 1998. The Syk family of protein tyrosine kinases in T-cell activation and development. Immunol. Rev. 165:167-80.
7. Geyra A., Z. Uni and D. Sklan 2001. Enterocyte dynamics and mucosal development in the posthatch chick. Poult. Sci. 80:776-82.
9. Honjo K. and Y. Hirota 1993. Immunohistochemical investigations of lymphocytes in the lymphoid organs of cyclophosphamide treated chickens. J. Vet. Med. Sci. 55:895-7.
10. Ishijima S. A., Y. X. Zeng, C. Kurashima, M. Utsuyama, T. Shirasawa, K. Sakamoto, and K. Hirokawa 1995. Expression of ZAP-70 gene in the developing thymus and various nonlymphoid tissues of embryonic and adult mice. Cell Immunol. 165:278-83.
14. Kuo C. T. and J. M. Leiden 1999. Transcriptional regulation of T lymphocyte development and function. Annu. Rev. Immunol. 17:149-87.
21. Nocentini G. and C. Riccardi 2005. GITR: a multifaceted regulator of immunity belonging to the tumor necrosis factor receptor superfamily. Eur. J. Immunol. 35:1016-22.
23. Rehli M., S. W. Krause, M. Kreutz and R. Andreesen 1995. Carboxypeptidase M is identical to the MAKI_antigen and its expression is associated with monocyte to macrophage differentiation. J. Biol. Chem. 270:15644-9.
24. Richter G. and S. Burdach 2004. ICOS: a new costimulatory ligand/receptor pair and its role in T-cell activion. Onkologie 27:91-5.
29. Sheela R. R., U. Babu, J. Mu, S. Elankumaran, D. A. Bautista, R. B. Rayboume, R. A. Heckert, and W. Song 2003. Immune responses against Salmonella enterica serovar Enteritidis infection in virally immunosuppressed chickens. Clin. Diagn. Lab. Immunol. 10:670-9.
30. Shin H. H., M. H. Lee, S. G. Kim, Y. H. Lee, B. S. Kwon, and H. S. Choi 2002. Recombinant glucocorticoid-induced tumor necrosis factor receptor (rGITR) induces NOS in murine macrophage. FEBS Lett. 514:275-80.
31. Smith M. W., M. A. Mitchell and M. A. Peacock 1990. Effects of genetic selection on growth rate and intestinal structure in the domestic fowl (Gallus domesticus). Comp Biochem Physiol A 97:57-63.
35. van Hemert S., A. J. Hoekman, M. A. Smits and J. M. J. Rebel 2004. Differences in intestinal gene expression profiles in broiler lines varying in susceptibility to malabsorption syndrome. Poult. Sci. 83:1675-1682.
36. Vreugdenhil A. C., A. M. Snoek, J. W. Greve and W. A. Buurman 2000. Lipopolysaccharide-binding protein is vectorially secreted and transported by cultured intestinal epithelial cells and is present in the intestinal mucus of mice. J. Immunol. 165:4561-6.
37. Wan Y. and M. W. Kirschner 2001. Identification of multiple CDH1 homologues in vertebrates conferring different substrate specificities. Proc. Natl. Acad. Sci. U.S.A. 98:13066-71.
39. Withanage G. S., P. Kaiser, P. Wigley, C. Powers, P. Mastroeni, H. Brooks, P. Barrow, A. Smith, D. Maskell, and I. McConnell 2004. Rapid expression of chemokines and proinflammatory cytokines in newly hatched chickens infected with Salmonella enterical serovar typhimurium. Infect. Immun. 72:2152-9.
40. Yoshida A., M. T. Minowa, S. Takamatsu, T. Hara, S. Oguri, H. Ikenaga, and M. Takeuchi 1999. Tissue-specific expression and chromosomal mapping of a human UDP-N-acetylglucosamine: alpha1, 3-d-mannoside beta1, 4-N-acetylglucosaminyltransferase. Glycobiology 9:303-10.

1. A set of genes or gene sequences comprising:
at least 20 genes selected from the group consisting of the genes depicted in Table 1, or a gene-specific fragment of any of said genes, and
at least five genes selected from the group consisting of Na/glucose transporter, K/Cl channel, I-FABP, L-FABP, Cytochrome P450, caspase, Beta-2-microglobin, guanylyn, calbindin, phosphatase, aldolase, actin, metalloproteinase, aminopeptidase, glycosaminotransferase, glutathion S transferase, maltase/glucoamylidase, sucrase/isomaltase, butyrophilin, apoB, cytochrome C oxidase, and a gene-specific fragment of any of said genes.

2. A method of determining a subject's intestinal health and/or disease state, said method comprising:
analyzing, in the subject, the set of genes or gene sequences of claim 1 so as to determine the intestinal health, and/or disease of the subject.

3. A method of detecting an intestinal disease in a subject, said method comprising:
taking a sample from the subject,
measuring, in the sample, expression levels of the set of genes or gene sequences of claim 1, and
comparing the expression levels with a reference value.

4. The method according to claim 3, wherein said sample is a bodily sample.

5. A method of measuring the increase of a subject's intestinal health status, said method comprising:
taking a series of samples of intestinal tissue from the subject, taken at different time points,
measuring, in the series of samples, expression levels of the set of genes or gene sequences of claim 1, and
comparing said expression levels with a reference value.

6. The method according to claim 3, further comprising:
measuring expression levels of at least two genes from the set of genes or gene sequences.

7. The method according to claim 6, comprising measuring expression levels of at least 30 genes or a gene-specific fragment of said genes.

8. The method according to claim 7, comprising measuring expression levels of at least 50 genes, or a gene-specific fragment of said genes.

9. The method according to claim 8, comprising measuring expression levels of at least 100 genes, or a gene-specific fragment of said genes.

10. The method according to claim 3, wherein a compound is administered to the subject before said sample is taken.

11. The method of claim 10 wherein the compound is selected from the group consisting of a food compound, a pathogenic compound, a virus, a microorganism, a pharmaceutical composition, and a part of any thereof.

12. A kit comprising a set of at least two oligonucleotide primers capable of specifically hybridizing to the set of genes or gene sequences of claim 1.

13. The kit of claim 12, wherein said genes are of porcine, avian, bovine, or human origin.

14. A kit comprising:
ingredients to measure protein levels of gene products encoded by the set of genes or gene sequences of claim 1.

15. The kit of claim 13, wherein said genes are of porcine, avian, bovine, or human origin.

16. The method according to claim 5, wherein a compound is administered to the subject before said sample is taken.

17. The method according to claim 6, wherein a compound is administered to the subject before said sample is taken.

18. The method according to claim 7, wherein a compound is administered to the subject before said sample is taken.

19. The method according to claim 8, wherein a compound is administered to the subject before said sample is taken.

20. The method according to claim 9, wherein a compound is administered to the subject before said sample is taken.
